SEQUENCE LISTING (29 sequences)

SEQ ID NO: 1 - DNA, 444 bp, Artificial Sequence
  HBV truncated core antigen gene
gacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtc

SEQ ID NO: 2 - protein, 148 aa, Artificial Sequence
  HBV truncated core antigen
DIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVV

SEQ ID NO: 3 - DNA, 447 bp, Artificial Sequence
  HBV truncated core antigen gene
atggacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatccaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggatccgcactccccctgcttatcgaccacccaacgcaccaattctgtccaccctgcccgagaccacagtggtc

SEQ ID NO: 4 - protein, 149 aa, Artificial Sequence
  HBV truncated core antigen
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVV

SEQ ID NO: 5 - DNA, 2529 bp, Artificial Sequence
  HBV pol antigen gene
atgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcacacgccacggggatgagtccttttgccagcagtctagtggcatcctgagcagatcccccgtggggccttgtctgcagtctcagctgcggaagagtagactgggactgcagccacagcagggacacctggcacgacggcagcagggaaggtctggcagtatccgggctagagtgcatcccacaactagaaggcctttcggcgtcgagccatcaggaagcggccacaccacaaacaccgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 6 - DNA, 2529 bp, Artificial Sequence
  HBV pol antigen gene
atgcccctgtcttaccagcactttagaaagctgctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcacacgccacggggatgagtccttttgccagcagtctagtggcatcctgagcagatcccccgtggggccttgtctgcagtctcagctgcggaagagtagactgggactgcagccacagcagggacacctggcacgacggcagcagggaaggtctggcagtatccgggctagagtgcatcccacaactagaaggcctttcggcgtcgagccatcaggaagcggccacaccacaaacaccgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 7 - protein, 843 aa, Artificial Sequence
  HBV pol antigen
MPLSYQHFRKLLLLDDEAGPLEEELPRLADEGLNRRVAEDLNLGNLNVSIPWTHKVGNFTGLYSSTVPVFNPEWQTPSFPNIHLQEDIINRCEQFVGPLTVNEKRRLKLIMPARFYPNVTKYLPLDKGIKPYYPEHLVNHYFQTRHYLHTLWKAGILYKRETTRSASFCGSPYSWEQELQHGRLVFQTSTRHGDESFCQQSSGILSRSPVGPCLQSQLRKSRLGLQPQQGHLARRQQGRSGSIRARVHPTTRRPFGVEPSGSGHTTNTASSSSSCLHQSAVRKAAYSHLSTSKRHSSSGHAVELHNIPPNSARSQSEGPVFSCWWLQFRNSKPCSDYCLSHIVNLLEDWGPCTEHGEHHIRIPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQNLHDSCSRNLYVSLLLLYKTFGRKLHLYSHPIILGFRKIPMGVGLSPFLLAQFTSAICSVVRRAFPHCLAFSYMNNVVLGAKSVQHLESLFTAVTNFLLSLGIHLNPNKTKRWGYSLNFMGYVIGSWGTLPQEHIVQKIKECFRKLPVNRPIDWKVCQRIVGLLGFAAPFTQCGYPALMPLYACIQSKQAFTFSPTYKAFLCKQYLNLYPVARQRPGLCQVFANATPTGWGLAIGHQRMRGTFVAPLPIHTAQLLAACFARSRSGAKLIGTDNSVVLSRKYTSFPWLLGCAANWILRGTSFVYVPSALNPADDPSRGRLGLYRPLLRLPFRPTTGRTSLYADSPSVPSHLPDRVHFASPLHVAWRPP

SEQ ID NO: 8 - DNA, 63 bp, Artificial Sequence
  Cystatin S signal peptide coding sequence
atggctcgacctctgtgtaccctgctactcctgatggctaccctggctggagctctggccagc

SEQ ID NO: 9 - protein, 21 aa, Artificial Sequence
  Cystatin S signal peptide sequence
MARPLCTLLLLMATLAGALAS

SEQ ID NO: 10 - DNA, 378 bp, Artificial Sequence
  triple enhancer regulatory sequence
ggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagac

SEQ ID NO: 11 - DNA, 12 bp, Artificial Sequence
  linker coding sequence
gccggagctggc

SEQ ID NO: 12 - DNA, 248 bp, Artificial Sequence
  ApoAI gene fragment
ttggccgtgctcttcctgacgggtaggtgtcccctaacctagggagccaaccatcggggggccttctccctaaatccccgtggcccaccctcctgggcagaggcagcaggtttctcactggccccctctcccccacctccaagcttggcctttcggctcagatctcagcccacagctggcctgatctgggtctcccctcccaccctcagggagccaggctcggcatttcgtcgacaagcttagccacc

SEQ ID NO: 13 - DNA, 130 bp, Artificial Sequence
  SV40 polyadenylation signal sequence
aacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 14 - DNA, 81 bp, Artificial Sequence
  immunoglobulin secretion signal coding sequence
atggagttcggcctgtcttgggtctttctggtggcaatcctgaagggcgtgcagtgtgaagtgcagctgctggagtctgga

SEQ ID NO: 15 - protein, 27 aa, Artificial Sequence
  immunoglobulin secretion signal sequence
MEFGLSWVFLVAILKGVQCEVQLLESG

SEQ ID NO: 16 - protein, 996 aa, Artificial Sequence
  HBV core-pol fusion antigen sequence
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVAGAGMPLSYQHFRKLLLLDDEAGPLEEELPRLADEGLNRRVAEDLNLGNLNVSIPWTHKVGNFTGLYSSTVPVFNPEWQTPSFPNIHLQEDIINRCEQFVGPLTVNEKRRLKLIMPARFYPNVTKYLPLDKGIKPYYPEHLVNHYFQTRHYLHTLWKAGILYKRETTRSASFCGSPYSWEQELQHGRLVFQTSTRHGDESFCQQSSGILSRSPVGPCLQSQLRKSRLGLQPQQGHLARRQQGRSGSIRARVHPTTRRPFGVEPSGSGHTTNTASSSSSCLHQSAVRKAAYSHLSTSKRHSSSGHAVELHNIPPNSARSQSEGPVFSCWWLQFRNSKPCSDYCLSHIVNLLEDWGPCTEHGEHHIRIPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQNLHDSCSRNLYVSLLLLYKTFGRKLHLYSHPIILGFRKIPMGVGLSPFLLAQFTSAICSVVRRAFPHCLAFSYMNNVVLGAKSVQHLESLFTAVTNFLLSLGIHLNPNKTKRWGYSLNFMGYVIGSWGTLPQEHIVQKIKECFRKLPVNRPIDWKVCQRIVGLLGFAAPFTQCGYPALMPLYACIQSKQAFTFSPTYKAFLCKQYLNLYPVARQRPGLCQVFANATPTGWGLAIGHQRMRGTFVAPLPIHTAQLLAACFARSRSGAKLIGTDNSVVLSRKYTSFPWLLGCAANWILRGTSFVYVPSALNPADDPSRGRLGLYRPLLRLPFRPTTGRTSLYADSPSVPSHLPDRVHFASPLHVAWRPP

SEQ ID NO: 17 - protein, 1023 aa, Artificial Sequence
  HBV core-pol fusion antigen sequence with Ig signal sequence
MEFGLSWVFLVAILKGVQCEVQLLESGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVAGAGMPLSYQHFRKLLLLDDEAGPLEEELPRLADEGLNRRVAEDLNLGNLNVSIPWTHKVGNFTGLYSSTVPVFNPEWQTPSFPNIHLQEDIINRCEQFVGPLTVNEKRRLKLIMPARFYPNVTKYLPLDKGIKPYYPEHLVNHYFQTRHYLHTLWKAGILYKRETTRSASFCGSPYSWEQELQHGRLVFQTSTRHGDESFCQQSSGILSRSPVGPCLQSQLRKSRLGLQPQQGHLARRQQGRSGSIRARVHPTTRRPFGVEPSGSGHTTNTASSSSSCLHQSAVRKAAYSHLSTSKRHSSSGHAVELHNIPPNSARSQSEGPVFSCWWLQFRNSKPCSDYCLSHIVNLLEDWGPCTEHGEHHIRIPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQNLHDSCSRNLYVSLLLLYKTFGRKLHLYSHPIILGFRKIPMGVGLSPFLLAQFTSAICSVVRRAFPHCLAFSYMNNVVLGAKSVQHLESLFTAVTNFLLSLGIHLNPNKTKRWGYSLNFMGYVIGSWGTLPQEHIVQKIKECFRKLPVNRPIDWKVCQRIVGLLGFAAPFTQCGYPALMPLYACIQSKQAFTFSPTYKAFLCKQYLNLYPVARQRPGLCQVFANATPTGWGLAIGHQRMRGTFVAPLPIHTAQLLAACFARSRSGAKLIGTDNSVVLSRKYTSFPWLLGCAANWILRGTSFVYVPSALNPADDPSRGRLGLYRPLLRLPFRPTTGRTSLYADSPSVPSHLPDRVHFASPLHVAWRPP

SEQ ID NO: 18 - DNA, 584 bp, Artificial Sequence
  hCMV promoter
tgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagc

SEQ ID NO: 19 - DNA, 684 bp, Artificial Sequence
  hCMV promoter sequence
accgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgg

SEQ ID NO: 20 - DNA, 225 bp, Artificial Sequence
  bGH polyA signal
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 21 - DNA, 671 bp, Artificial Sequence
  pUC ORI
cccgtagaaaagatcaaaggatcttcttgagatcctttttttctgcgcgtaatctgctgcttgcaaacaaaaaaaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacat

SEQ ID NO: 22 - DNA, 795 bp, Artificial Sequence
  KanR coding sequence
atgattgagcaagatggtcttcacgctggctcgccagctgcgtgggtggaacgcctgtttggttatgattgggcgcagcagactattggatgttccgacgcggctgtatttcggctgtctgctcagggtcgccccgtgctgtttgtgaagacggatttgtctggcgcattaaatgagttacaggacgaggcggctcgtctgagttggttggccaccaccggcgtgccctgcgccgcagtgctggatgtcgtgacagaagcaggccgcgattggctccttctcggcgaagtgccgggccaggacctgctcagcagccacttggcaccggcagaaaaagtttctatcatggccgacgccatgcgtcgtcttcacactctcgatccggccacgtgcccctttgaccaccaggccaagcatcgtattgaacgtgcgcgtactcggatggaagcaggtttagtagaccaggacgatttggatgaggaacatcaaggcctggccccggctgaactgtttgcgcgcttaaaagcgtcgatgccagatggcgaagatttggtagtcacccatggagatgcgtgtttgccaaacatcatggttgaaaatggccgcttctcaggctttattgactgtgggcgcctgggtgttgccgaccgctatcaagatattgcgctcgcaactcgtgacatcgctgaagagctgggcggagaatgggctgaccgtttcctggtactgtatggcattgcagcgcccgattcccaacgcatcgcattttatcgtctgctggatgagtttttctaa

SEQ ID NO: 23 - protein, 264 aa, Artificial Sequence
  Codon optimized Kanr
MIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 24 - DNA, 99 bp, Artificial Sequence
  bla promoter
acccctatttgtttatttttctaaatacattcaaatatgtatccgctcatgagacaataaccctgataaatgcttcaataatattgaaaaaggaagagt

SEQ ID NO: 25 - DNA, 3377 bp, Artificial sequence
  Viral genome sequence
gcgcaccggggatcctaggctttttggattgcgctttcctctagatcaactgggtgtcaggccctatcctacagaaggatgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatgcacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcctcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgaagaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgaccaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttgtcttcatactccctcgaagcttccctggtcatttcaacatcgataagcttaatgtccttcctattctgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgaattgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagaggtcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 26 - DNA, 7229 bp, Artificial sequence
  Viral genome sequence
gcgcaccggggatcctaggcgtttagttgcgctgtttggttgcacaactttcttcgtgaggctgtcagaagtggacctggctgatagcgatgggtcaaggcaagtccagagaggagaaaggcaccaatagtacaaacagggccgaaatcctaccagataccacctatcttggccctttaagctgcaaatcttgctggcagaaatttgacagcttggtaagatgccatgaccactacctttgcaggcactgtttaaaccttctgctgtcagtatccgacaggtgtcctctttgtaaatatccattaccaaccagattgaagatatcaacagccccaagctctccacctccctacgaagagtaacaccgtccggccccggccccgacaaacagcccagcacaagggaaccgcacgtcacccaacgcacacagacacagcacccaacacagaacacgcacacacacacacacacacacccacacgcacgcgcccccaccaccggggggcgcccccccccggggggcggccccccgggagcccgggcggagccccacggagatgcccatcagtcgatgtcctcggccaccgacccgcccagccaatcgtcgcaggacctccccttgagtctaaacctgccccccactgtttcatacatcaaagtgctcctagatttgctaaaacaaagtctgcaatccttaaaggcgaaccagtctggcaaaagcgacagtggaatcagcagaatagatctgtctatacatagttcctggaggattacacttatctctgaacccaacaaatgttcaccagttctgaatcgatgcaggaagaggttcccaaggacatcactaatcttttcatagccctcaagtcctgctagaaagactttcatgtccttggtctccagcttcacaatgatattttggacaaggtttcttccttcaaaaagggcacccatctttacagtcagtggcacaggctcccactcaggtccaactctctcaaagtcaatagatctaatcccatccagtattcttttggagcccaacaactcaagctcaagagaatcaccaagtatcaagggatcttccatgtaatcctcaaactcttcagatctgatatcaaagacaccatcgttcaccttgaagacagagtctgtcctcagtaagtggaggcattcatccaacattcttctatctatctcacccttaaagaggtgagagcatgataaaagttcagccacacctggattctgtaattggcacctaaccaagaatatcaatgaaaatttccttaaacagtcagtattattctgattgtgcgtaaagtccactgaaattgaaaactccaataccccttttgtgtagttgagcatgtagtcccacagatcctttaaggatttaaatgcctttgggtttgtcaggccctgcctaatcaacatggcagcattacacacaacatctcccattcggtaagagaaccacccaaaaccaaactgcaaatcattcctaaacataggcctctccacatttttgttcaccacctttgagacaaatgattgaaaggggcccagtgcctcagcaccatcttcagatggcatcatttctttatgagggaaccatgaaaaattgcctaatgtcctggttgttgcaacaaattctcgaacaaatgattcaaaatacacctgttttaagaagttcttgcagacatccctcgtgctaacaacaaattcatcaaccagactggagtcagatcgctgatgagaattggcaaggtcagaaaacagaacagtgtaatgttcatcccttttccacttaacaacatgagaaatgagtgacaaggattctgagttaatatcaattaaaacacagaggtcaaggaatttaattctgggactccacctcatgttttttgagctcatgtcagacataaatggaagaagctgatcctcaaagatcttgggatatagccgcctcacagattgaatcacttggttcaaattcactttgtcctccagtagccttgagctctcaggctttcttgctacataatcacatgggtttaagtgcttaagagttaggttctcactgttattcttccctttggtcggttctgctaggacccaaacacccaactcaaaagagttgctcaatgaaatacaaatgtagtcccaaagaagaggccttaaaaggcatatatgatcacggtgggcttctggatgagactgtttgtcacaaatgtacagcgttataccatcccgattgcaaactcttgtcacatgatcatctgtggttagatcctcaagcagctttttgatatacagattttccctatttttgtttctcacacacctgcttcctagagttttgcaaaggcctataaagccagatgagatacaactctggaaagctgacttgttgattgcttctgacagcagcttctgtgcaccccttgtgaatttactacaaagtttgttctggagtgtcttgatcaatgatgggattctttcctcttggaaagtcatcactgatggataaaccaccttttgtcttaaaaccatccttaatgggaacatttcattcaaattcaaccagttaacatctgctaactgattcagatcttcttcaagaccgaggaggtctcccaattgaagaatggcctcctttttatctctgttaaataggtctaagaaaaattcttcattaaattcaccatttttgagcttatgatgcagtttccttacaagctttcttacaacctttgtttcattaggacacagttcctcaatgagtctttgtattctgtaacctctagaaccatccagccaatctttcacatcagtgttggtattcagtagaaatggatccaaagggaaattggcatactttaggaggtccagtgttctcctttggatactattaactagggagactgggacgccatttgcgatggcttgatctgcaattgtatctattgtttcacaaagttgatgtggctctttacacttgacattgtgtagcgctgcagatacaaactttgtgagaagagggacttcctccccccatacatagaatctagatttaaattctgcagcgaacctcccagccacactttttgggctgataaatttgtttaacaagccgctcagatgagattggaattccaacaggacaaggacttcctccggatcacttacaaccaggtcactcagcctcctatcaaataaagtgatctgatcatcacttgatgtgtaagcctctggtctttcgccaaagataacaccaatgcagtagttgatgaacctctcgctaagcaaaccatagaagtcagaagcattatgcaagattccctgccccatatcaataaggctggatatatgggatggcactatccccatttcaaaatattgtctgaaaattctctcagtaacagttgtttctgaacccctgagaagttttagcttcgacttgacatatgatttcatcattgcattcacaacaggaaaggggacctcgacaagcttatgcatgtgccaagttaacaaagtgctaacatgatctttcccggaacgcacatactggtcatcacctagtttgagattttgtagaaacattaagaacaaaaatgggcacatcattggtccccatttgctgtgatccatactatagtttaagaacccttcccgcacattgatagtcattgacaagattgcattttcaaattccttatcattgtttaaacaggagcctgaaaagaaacttgaaaaagactcaaaataatcttctattaaccttgtgaacatttttgtcctcaaatctccaatatagagttctctatttcccccaacctgctctttataagatagtgcaaatttcagccttccagagtcaggacctactgaggtgtatgatgttggtgattcttctgagtagaagcacagatttttcaaagcagcactcatacattgtgtcaacgacagagctttactaagggactcagaattactttccctctcactgattctcacgtcttcttccagtttgtcccagtcaaatttgaaattcaagccttgcctttgcatatgcctgtatttccctgagtacgcatttgcattcatttgcaacagaatcatcttcatgcaagaaaaccaatcattctcagaaaagaactttctacaaaggttttttgccatctcatcgaggccacactgatctttaatgactgaggtgaaatacaaaggtgacagctctgtggaaccctcaacagcctcacagataaatttcatgtcatcattggttagacatgatgggtcaaagtcttctactaaatggaaagatatttctgacaagataacttttcttaagtgagccatcttccctgttagaataagctgtaaatgatgtagtccttttgtatttgtaagtttttctccatctcctttgtcattggccctcctacctcttctgtaccgtgctattgtggtgttgaccttttcttcgagacttttgaagaagcttgtctcttcttctccatcaaaacatatttctgccaggttgtcttccgatctccctgtctcttctcccttggaaccgatgaccaatctagagactaacttggaaactttatattcatagtctgagtggctcaacttatacttttgttttcttacgaaactctccgtaatttgactcacagcactaacaagcaatttgttaaagtcatattccagaagtcgttctccatttagatgcttattaaccaccacacttttgttactagcaagatctaatgctgtcgcacatccagagttagtcatgggatctaggctgtttagcttcttctctcctttgaaaattaaagtgccgttgttaaatgaagacaccattaggctaaaggcttccagattaacacctggagttgtatgctgacagtcaatttctttactagtgaatctcttcatttgctcatagaacacacattcttcctcaggagtgattgcttccttggggttgacaaaaaaaccaaattgacttttgggctcaaagaacttttcaaaacattttatctgatctgttagcctgtcaggggtctcctttgtgatcaaatgacacaggtatgacacattcaacataaatttaaattttgcactcaacaacaccttctcaccagtaccaaaaatagtttttattaggaatctaagcagcttatacaccaccttctcagcaggtgtgatcagatcctccctcaacttatccattaatgatgtagatgaaaaatctgacactattgccatcaccaaatatctgacactctgtacctgcttttgatttctctttgttgggttggtgagcattagcaacaatagggtcctcagtgcaacctcaatgtcggtgagacagtctttcaaatcaggacatgatctaatccatgaaatcatgatgtctatcatattgtataagacctcatctgaaaaaattggtaaaaagaaccttttaggatctgcatagaaggaaattaaatgaccatccgggccttgtatggagtagcaccttgaagattctccagtcttctggtataataggtggtattcttcagagtccagttttattacttggcaaaacacttctttgcattctaccacttgatatctcacagaccctatttgattttgccttagtctagcaactgagctagttttcatactgtttgttaaggccagacaaacagatgataatcttctcaggctctgtatgttcttcagctgctctgtgctgggttggaaattgtaatcttcaaacttcgtataatacattatcgggtgagctccaattttcataaagttctcaaattcagtgaatggtatgtggcattcttgctcaaggtgttcagacagtccgtaatgctcgaaactcagtcccaccactaacaggcatttttgaatttttgcaatgaactcactaatagatgccctaaacaattcctcaaaagacacctttctaaacacctttgacttttttctattcctcaaaagtctaatgaactcctctttagtgctgtgaaagcttaccagcctatcattcacactactatagcaacaacccacccagtgtttatcattttttaaccctttgaatttcgactgttttatcaatgaggaaagacacaaaacatccagatttaacaactgtctccttctagtattcaacagtttcaaactcttgactttgtttaacatagagaggagcctctcatattcagtgctagtctcacttcccctttcgtgcccatgggtctctgcagttatgaatctcatcaaaggacaggattcgactgcctccctgcttaatgttaagatatcatcactatcagcaaggttttcatagagctcagagaattccttgatcaagccttcagggtttactttctgaaagtttctctttaatttcccactttctaaatctcttctaaacctgctgaaaagagagtttattccaaaaaccacatcatcacagctcatgttggggttgatgccttcgtggcacatcctcataatttcatcattgtgagttgacctcgcatctttcagaattttcatagagtccataccggagcgcttgtcgatagtagtcttcagggactcacagagtctaaaatattcagactcttcaaagactttctcattttggttagaatactccaaaagtttgaataaaaggtctctaaatttgaagtttgcccactctggcataaaactattatcataatcacaacgaccatctactattggaactaatgtgacacccgcaacagcaaggtcttccctgatgcatgccaatttgttagtgtcctctataaatttcttctcaaaactggctggagtgctcctaacaaaacactcaagaagaatgagagaattgtctatcagcttgtaaccatcaggaatgataagtggtagtcctgggcatacaattccagactccaccaaaattgtttccacagacttatcgtcgtggttgtgtgtgcagccactcttgtctgcactgtctatttcaatgcagcgtgacagcaacttgagtccctcaatcagaaccattctgggttccctttgtcccagaaagttgagtttctgccttgacaacctctcatcctgttctatatagtttaaacataactctctcaattctgagatgatttcatccattgcgcatcaaaaagcctaggatcctcggtgcg

SEQ ID NO: 27 - DNA, 7205 bp, Artificial sequence
  Viral genome sequence
gcgcaccggggatcctaggcatttttgttgcgcattttgttgtgttatttgttgcacagcccttcatcgtgggaccttcacaaacaaaccaaaccaccagccatgggccaaggcaagtccaaagagggaagggatgccagcaatacgagcagagctgaaattctgccagacaccacctatctcggacctctgaactgcaagtcatgctggcagagatttgacagtttagtcagatgccatgaccactatctctgcagacactgcctgaacctcctgctgtcagtctccgacaggtgccctctctgcaaacatccattgccaaccaaactgaaaatatccacggccccaagctctccacccccttacgaggagtgacgccccgagccccaacaccgacacaaggaggccaccaacacaacgcccaacacggaacacacacacacacacccacacacacatccacacacacgcgcccccacaacgggggcgcccccccgggggtggccccccgggtgctcgggcggagccccacggagaggccaattagtcgatctcctcgaccaccgacttggtcagccagtcatcacaggacttgcccttaagtctgtacttgcccacaactgtttcatacatcaccgtgttctttgacttactgaaacatagcctacagtctttgaaagtgaaccagtcaggcacaagtgacagcggtaccagtagaatggatctatctatacacaactcttggagaattgtgctaatttccgacccctgtagatgctcaccagttctgaatcgatgtagaagaaggctcccaaggacgtcatcaaaatttccataaccctcgagctctgccaagaaaactctcatatccttggtctccagtttcacaacgatgttctgaacaaggcttcttccctcaaaaagagcacccattctcacagtcaagggcacaggctcccattcaggcccaatcctctcaaaatcaagggatctgatcccgtccagtattttccttgagcctatcagctcaagctcaagagagtcaccgagtatcagggggtcctccatatagtcctcaaactcttcagacctaatgtcaaaaacaccatcgttcaccttgaagatagagtctgatctcaacaggtggaggcattcgtccaagaaccttctgtccacctcacctttaaagaggtgagagcatgataggaactcagctacacctggaccttgtaactggcacttcactaaaaagatcaatgaaaacttcctcaaacaatcagtgttattctggttgtgagtgaaatctactgtaattgagaactctagcactccctctgtattatttatcatgtaatcccacaagtttctcaaagacttgaatgcctttggatttgtcaagccttgtttgattagcatggcagcattgcacacaatatctcccaatcggtaagagaaccatccaaatccaaattgcaagtcattcctaaacatgggcctctccatatttttgttcactacttttaagatgaatgattggaaaggccccaatgcttcagcgccatcttcagatggcatcatgtctttatgagggaaccatgaaaaacttcctagagttctgcttgttgctacaaattctcgtacaaatgactcaaaatacacttgttttaaaaagtttttgcagacatcccttgtactaacgacaaattcatcaacaaggcttgagtcagagcgctgatgggaatttacaagatcagaaaatagaacagtgtagtgttcgtccctcttccacttaactacatgagaaatgagcgataaagattctgaattgatatcgatcaatacgcaaaggtcaaggaatttgattctgggactccatctcatgttttttgagctcatatcagacatgaagggaagcagctgatcttcatagattttagggtacaatcgcctcacagattggattacatggtttaaacttatcttgtcctccagtagccttgaactctcaggcttccttgctacataatcacatgggttcaagtgcttgaggcttgagcttccctcattcttccctttcacaggttcagctaagacccaaacacccaactcaaaggaattactcagtgagatgcaaatatagtcccaaaggaggggcctcaagagactgatgtggtcgcagtgagcttctggatgactttgcctgtcacaaatgtacaacattatgccatcatgtctgtggattgctgtcacatgcgcatccatagctagatcctcaagcacttttctaatgtatagattgtccctatttttatttctcacacatctacttcccaaagttttgcaaagacctataaagcctgatgagatgcaactttgaaaggctgacttattgattgcttctgacagcaacttctgtgcacctcttgtgaacttactgcagagcttgttctggagtgtcttgattaatgatgggattctttcctcttggaaagtcattactgatggataaaccactttctgcctcaagaccattcttaatgggaacaactcattcaaattcagccaatttatgtttgccaattgacttagatcctcttcgaggccaaggatgtttcccaactgaagaatggcttcctttttatccctattgaagaggtctaagaagaattcttcattgaactcaccattcttgagcttatgatgtagtctccttacaagccttctcatgaccttcgtttcactaggacacaattcttcaataagcctttggattctgtaacctctagagccatccaaccaatccttgacatcagtattagtgttaagcaaaaatgggtccaagggaaagttggcatattttaagaggtctaatgttctcttctggatgcagtttaccaatgaaactggaacaccatttgcaacagcttgatcggcaattgtatctattgtttcacagagttggtgtggctctttacacttaacgttgtgtaatgctgctgacacaaattttgttaaaagtgggacctcttccccccacacataaaatctggatttaaattctgcagcaaatcgccccaccacacttttcggactgatgaacttgttaagcaagccactcaaatgagaatgaaattccagcaatacaaggacttcctcagggtcactatcaaccagttcactcaatctcctatcaaataaggtgatctgatcatcacttgatgtgtaagattctggtctctcaccaaaaatgacaccgatacaataattaatgaatctctcactgattaagccgtaaaagtcagaggcattatgtaagattccctgtcccatgtcaatgagactgcttatatgggaaggcactattcctaattcaaaatattctcgaaagattctttcagtcacagttgtctctgaacccctaagaagtttcagctttgatttgatatatgatttcatcattgcattcacaacaggaaaagggacctcaacaagtttgtgcatgtgccaagttaataaggtgctgatatgatcctttccggaacgcacatactggtcatcacccagtttgagattttgaaggagcattaaaaacaaaaatgggcacatcattggcccccatttgctatgatccatactgtagttcaacaacccctctcgcacattgatggtcattgatagaattgcattttcaaattctttgtcattgtttaagcatgaacctgagaagaagctagaaaaagactcaaaataatcctctatcaatcttgtaaacatttttgttctcaaatccccaatataaagttctctgtttcctccaacctgctctttgtatgataacgcaaacttcaaccttccggaatcaggaccaactgaagtgtatgacgttggtgactcctctgagtaaaaacataaattctttaaagcagcactcatgcattttgtcaatgatagagccttacttagagactcagaattactttccctttcactaattctaacatcttcttctagtttgtcccagtcaaacttgaaattcagaccttgtctttgcatgtgcctgtatttccctgagtatgcatttgcattcatttgcagtagaatcattttcatacacgaaaaccaatcaccctctgaaaaaaacttcctgcagaggttttttgccatttcatccagaccacattgttctttgacagctgaagtgaaatacaatggtgacagttctgtagaagtttcaatagcctcacagataaatttcatgtcatcattggtgagacaagatgggtcaaaatcttccacaagatgaaaagaaatttctgataagatgaccttccttaaatatgccattttacctgacaatatagtctgaaggtgatgcaatccttttgtattttcaaaccccacctcattttccccttcattggtcttcttgcttctttcataccgctttattgtggagttgaccttatcttctaaattcttgaagaaacttgtctcttcttccccatcaaagcatatgtctgctgagtcaccttctagtttcccagcttctgtttctttagagccgataaccaatctagagaccaactttgaaaccttgtactcgtaatctgagtggttcaatttgtacttctgctttctcatgaagctctctgtgatctgactcacagcactaacaagcaatttgttaaaatcatactctaggagccgttccccatttaaatgtttgttaacaaccacacttttgttgctggcaaggtctaatgctgttgcacacccagagttagtcatgggatccaagctattgagcctcttctcccctttgaaaatcaaagtgccattgttgaatgaggacaccatcatgctaaaggcctccagattgacacctggggttgtgcgctgacagtcaacttctttcccagtgaacttcttcatttggtcataaaaaacacactcttcctcaggggtgattgactctttagggttaacaaagaagccaaactcacttttaggctcaaagaatttctcaaagcatttaatttgatctgtcagcctatcaggggtttcctttgtgattaaatgacacaggtatgacacattcaacatgaacttgaactttgcgctcaacagtaccttttcaccagtcccaaaaacagttttgatcaaaaatctgagcaatttgtacactactttctcagcaggtgtgatcaaatcctccttcaacttgtccatcaatgatgtggatgagaagtctgagacaatggccatcactaaatacctaatgttttgaacctgtttttgattcctctttgttgggttggtgagcatgagtaataatagggttctcaatgcaatctcaacatcatcaatgctgtccttcaagtcaggacatgatctgatccatgagatcatggtgtcaatcatgttgtgcaacacttcatctgagaagattggtaaaaagaacctttttgggtctgcataaaaagagattagatggccattgggaccttgtatagaataacaccttgaggattctccagtcttttgatacagcaggtgatattcctcagagtccaattttatcacttggcaaaatacctctttacattccaccacttgataccttacagagcccaattggttttgtcttaatctagcaactgaacttgttttcatactgtttgtcaaagctagacagacagatgacaatcttttcaaactatgcatgttccttaattgttccgtattaggctggaaatcataatcttcaaactttgtataatacattataggatgagttccggacctcatgaaattctcaaactcaataaatggtatgtggcactcatgctcaagatgttcagacagaccatagtgcccaaaactaagtcccaccactgacaagcacctttgaacttttaaaatgaactcatttatggatgttctaaacaaatcctcaagagatacctttctatacgcctttgactttctcctgttccttagaagtctgatgaactcttccttggtgctatgaaagctcaccaacctatcattcacactcccatagcaacaaccaacccagtgcttatcattttttgaccctttgagtttagactgtttgatcaacgaagagagacacaagacatccaaattcagtaactgtctccttctggtgttcaataattttaaacttttaactttgttcaacatagagaggagcctctcatactcagtgctagtctcacttcctctctcataaccatgggtatctgctgtgataaatctcatcaaaggacaggattcaactgcctccttgcttagtgctgaaatgtcatcactgtcagcaagagtctcataaagctcagagaattccttaattaaatttccggggttgattttctgaaaactcctcttgagcttcccagtttccaagtctcttctaaacctgctgtaaagggagtttatgccaagaaccacatcatcgcagttcatgtttgggttgacaccatcatggcacattttcataatttcatcattgtgaaatgatcttgcatctttcaagattttcatagagtctataccggaacgcttatcaacagtggtcttgagagattcgcaaagtctgaagtactcagattcctcaaagactttctcatcttggctagaatactctaaaagtttaaacagaaggtctctgaacttgaaattcacccactctggcataaagctgttatcataatcacaccgaccatccactattgggaccaatgtgatacccgcaatggcaaggtcttctttgatacaggctagtttattggtgtcctctataaatttcttctcaaaactagctggtgtgcttctaacgaagcactcaagaagaatgagggaattgtcaatcagtttataaccatcaggaatgatcaaaggcagtcccgggcacacaatcccagactctattagaattgcctcaacagatttatcatcatggttgtgtatgcagccgctcttgtcagcactgtctatctctatacaacgcgacaaaagtttgagtccctctatcaataccattctgggttctctttgccctaaaaagttgagcttctgccttgacaacctctcatcttgttctatgtggtttaagcacaactctctcaactccgaaatagcctcatccattgcgcatcaaaaagcctaggatcctcggtgcg

SEQ ID NO: 28 - DNA, 3359 bp, Artificial sequence
  viral genome sequence
cgcaccggggatcctaggctttttggattgcgctttcctcagctccgtcttgtgggagaatgggtcaaattgtgacgatgtttgaggctctgcctcacatcattgatgaggtcattaacattgtcattatcgtgcttattatcatcacgagcatcaaagctgtgtacaatttcgccacctgcgggatacttgcattgatcagctttctttttctggctggcaggtcctgtggaatgtatggtcttgatgggcctgacatttacaaaggggtttaccgattcaagtcagtggagtttgacatgtcttaccttaacctgacgatgcccaatgcatgttcggcaaacaactcccatcattatataagtatggggacttctggattggagttaaccttcacaaatgactccatcatcacccacaacttttgtaatctgacttccgccctcaacaagaggacttttgaccacacacttatgagtatagtctcaagtctgcacctcagcattagaggggtccccagctacaaagcagtgtcctgtgattttaacaatggcatcactattcaatacaacctgtcattttctaatgcacagagcgctctgagtcaatgtaagaccttcagggggagagtcctggatatgttcagaactgcttttggaggaaagtacatgaggagtggctggggctggacaggttcagatggcaagactacttggtgcagccagacaaactaccaatatctgattatacaaaacaggacttgggaaaaccactgcaggtacgcaggccctttcggaatgtctagaattctcttcgctcaagaaaagacaaggtttctaactagaaggcttgcaggcacattcacttggactttatcagactcatcaggagtggagaatccaggtggttactgcttgaccaagtggatgatcctcgctgcagagctcaagtgttttgggaacacagctgttgcaaagtgcaatgtaaatcatgatgaagagttctgtgatatgctacgactgattgattacaacaaggctgctttgagtaaattcaaagaagatgtagaatccgctctacatctgttcaagacaacagtgaattctttgatttctgatcagcttttgatgagaaatcacctaagagacttgatgggagtgccatactgcaattactcgaaattctggtatctagagcatgcaaagactggtgagactagtgtccccaagtgctggcttgtcagcaatggttcttatttgaatgaaacccatttcagcgaccaaattgagcaggaagcagataatatgatcacagaaatgctgagaaaggactacataaaaaggcaagggagtacccctctagccttgatggatctattgatgttttctacatcagcatatttgatcagcatctttctgcatcttgtgaggataccaacacacagacacataaagggcggctcatgcccaaaaccacatcggttaaccagcaagggaatctgtagttgtggtgcatttaaagtaccaggtgtggaaaccacctggaaaagacgctgaacagcagcgcctccctgactcaccacctcgaaagaggtggtgagtcagggaggcccagagggtcttagagtgttacgacatttggacctctgaagattaggtcatgtggtaggatattgtggacagttttcaggtcggggagccttgccttggaggcgctttcaaagatgatacagtccatgagtgcacagtgtggggtgacctctttctttttcttgtccctcactattccagtgtgcatcttgcatagccagccatatttgtcccagactttgtcctcatattctcttgaagcttctttagtcatctcaacatcgatgagcttaatgtctcttctgttttgtgaatctaggagtttcctgatgtcatcagatccctgacaacttaggaccattccctgtggaagagcacctattactgaagatgtcagcccaggttgtgcattgaagaggtcagcaaggtccatgccatgtgagtatttggagtcctgcttgaattgtttttgatcagtgggttctctatagaaatgtatgtactgcccattctgtggctgaaatattgctatttctaccgggtcattaaatctgccctcaatgtcaatccatgtaggagcgttagggtcaatacctcccatgaggtccttcagcaacattgtttggctgtagcttaagcccacctgaggtgggcccgctgccccaggcgctggtttgggtgagttggccataggcctctcatttgtcagatcaattgttgtgttctcccatgctctccctacaactgatgttctacaagctatgtatggccacccctcccctgaaagacagactttgtagaggatgttctcgtaaggattcctgtctccaacctgatcagaaacaaacatgttgagtttcttcttggccccaagaactgctttcaggagatcctcactgttgcttggcttaattaagatggattccaacatgttacccccatctaacaaggctgcccctgctttcacagcagcaccgagactgaaattgtagccagatatgttgatgctagactgctgctcagtgatgactcccaagactgggtgcttgtctttcagcctttcaaggtcacttaggttcgggtacttgactgtgtaaagcagcccaaggtctgtgagtgcttgcacaacgtcattgagtgaggtttgtgattgtttggccatacaagccattgttaagcttggcattgtgccgaattgattgttcagaagtgatgagtccttcacatcccagaccctcaccacaccatttgcactctgctgaggtctcctcattccaaccatttgcagaatctgagatctttggtcaagctgttgtgctgttaagttccccatgtagactccagaagttagaggcctttcagacctcatgattttagccttcagtttttcaaggtcagctgcaagggacatcagttcttctgcactaagcctccctacttttagaacattcttttttgatgttgactttaggtccacaagggaatacacagtttggttgaggcttctgagtctctgtaaatctttgtcatccctcttctctttcctcatgatcctctgaacattgctcacctcagagaagtctaatccattcagaaggctggtggcatccttgatcacagcagctttcacatctgatgtgaagccttgaagctctctcctcaatgcctgggtccattgaaagcttttaacttctttggacagagacattttgtcactcagtggatttccaagtcaaatgcgcaatcaaaatgcctaggatccactgtgcg

SEQ ID NO: 29 - DNA, 3376 bp, Artificial sequence
  Viral genome sequence
cgcaccggggatcctaggctttttggattgcgctttcctctagatcaactgggtgtcaggccctatcctacagaaggatgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatcgacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcttcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgccgaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgatcaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttgtcttcatactccctcgaagcttccctggtcatttcaacatcgataagcttaatgtccttcctattctgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgaattgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagaggtcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

(19) United States
(12) Patent Application Publication
(10) Pub. No.: US 2022/0226467 A1
(43) Pub. Date: Jul. 21, 2022

(54) ARENAVIRUS VECTORS FOR HEPATITIS B VIRUS (HBV) VACCINES AND USES THEREOF

(71) Applicant: Janssen Sciences Ireland Unlimited Company, Co Cork (IE)

(72) Inventors: Helen HORTON, Beerse (BE); Daniel BODEN, Beerse (BE)

(21) Appl. No.: 17/596,750

(22) PCT Filed: Jun. 18, 2020
(86) PCT No.: PCT/IB2020/055719

§ 371 Date: Dec. 17, 2021

Related U.S. Application Data
(60) Provisional application No. 62/862,813, filed on Jun. 18, 2019.

(51) Int. Cl. A61K 39/29; C12N 15/86; C07K 16/08

(52) U.S. Cl. CPC A61K 39/292; A61K 2039/5256; C07K 16/082; C12N 15/86

(57) ABSTRACT

Arenavirus vectors encoding hepatitis B virus (HBV) vaccines are described. Methods of inducing an immune response against HBV or treating an HBV-induced disease, particularly in individuals having chronic HBV infection, using the disclosed arenavirus vectors are also described.

### CROSS REFERENCE TO RELATED APPLICATION

This application claims priority to U.S. Provisional Application No. 62/862,813 filed on Jun. 18, 2019, the disclosure of which is incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “065814.11194/11WO1 Sequence Listing” and a creation date of Jun. 10, 2020 and having a size of 77.3 kb. The sequence listing submitted via EFS-Web is part of the specification and is herein incorporated by reference in its entirety.

### BACKGROUND OF THE INVENTION

Hepatitis B virus (HBV) is a small 3.2-kb hepatotropic DNA virus that encodes four open reading frames and seven proteins. Approximately 240 million people have chronic hepatitis B infection (chronic HBV), characterized by persistent virus and subvirus particles in the blood for more than 6 months (Cohen et al. J. Viral Hepat. (2011) 18(6), 377-83). Persistent HBV infection leads to T-cell exhaustion in circulating and intrahepatic HBV-specific CD4+ and CD8+ T-cells through chronic stimulation of HBV-specific T-cell receptors with viral peptides and circulating antigens. As a result, T-cell polyfunctionality is decreased (i.e., decreased levels of IL-2, tumor necrosis factor (TNF)-α, IFN-γ, and lack of proliferation).

A safe and effective prophylactic vaccine against HBV infection has been available since the 1980s and is the mainstay of hepatitis B prevention (World Health Organization, Hepatitis B: Fact sheet No. 204 [Internet] 2015 March.). The World Health Organization recommends vaccination of all infants, and, in countries where there is low or intermediate hepatitis B endemicity, vaccination of all children and adolescents (<18 years of age), and of people of certain at risk population categories. Due to vaccination, worldwide infection rates have dropped dramatically. However, prophylactic vaccines do not cure established HBV infection.

Chronic HBV is currently treated with IFN-α and nucleoside or nucleotide analogs, but there is no ultimate cure due to the persistence in infected hepatocytes of an intracellular viral replication intermediate called covalently closed circular DNA (cccDNA), which plays a fundamental role as a template for viral RNAs, and thus new virions. It is thought that induced virus-specific T-cell and B-cell responses can effectively eliminate cccDNA-carrying hepatocytes. Current therapies targeting the HBV polymerase suppress viremia, but offer limited effect on cccDNA that resides in the nucleus and related production of circulating antigen. The most rigorous form of a cure can be elimination of HBV cccDNA from the organism, which has neither been observed as a naturally occurring outcome nor as a result of any therapeutic intervention. However, loss of HBV surface antigens (HBsAg) is a clinically credible equivalent of a cure, since disease relapse can occur only in cases of severe immunosuppression, which can then be prevented by prophylactic treatment. Thus, at least from a clinical standpoint, loss of HBsAg is associated with the most stringent form of immune reconstitution against HBV.

For example, immune modulation with pegylated interferon (pegIFN)-α has proven better in comparison to nucleoside or nucleotide therapy in terms of sustained off-treatment response with a finite treatment course. Besides a direct antiviral effect, IFN-α is reported to exert epigenetic suppression of cccDNA in cell culture and humanized mice, which leads to reduction of virion productivity and transcripts (Belloni et al. J. Clin. Invest. (2012) 122(2), 529-537). However, this therapy is still fraught with side-effects and overall responses are rather low, in part because IFN-a has only poor modulatory influences on HBV-specific T-cells. In particular, cure rates are low (<10%) and toxicity is high. Likewise, direct acting HBV antivirals, namely the HBV polymerase inhibitors entecavir and tenofovir, are effective as monotherapy in inducing viral suppression with a high genetic barrier to emergence of drug resistant mutants and consecutive prevention of liver disease progression. However, cure of chronic hepatitis B, defined by HBsAg loss or seroconversion, is rarely achieved with such HBV polymerase inhibitors. Therefore, these antivirals in theory need to be administered indefinitely to prevent reoccurrence of liver disease, similar to antiretroviral therapy for human immunodeficiency virus (HIV).

Therapeutic vaccination has the potential to eliminate HBV from chronically infected patients (Michel et al. J. Hepatol. (2011) 54(6), 1286-1296). Many strategies have been explored, but to date therapeutic vaccination has not proven successful.

### BRIEF SUMMARY OF THE INVENTION

Accordingly, there is an unmet medical need in the treatment of hepatitis B virus (HBV), particularly chronic HBV, for a finite well-tolerated treatment with a higher cure rate. The invention satisfies this need by providing therapeutic compositions and methods for inducing an immune response against hepatitis B viruses (HBV) infection. The immunogenic compositions/combinations and methods of the invention can be used to provide therapeutic immunity to a subject, such as a subject having chronic HBV infection.

In a general aspect, the application relates to an arenavirus vector comprising one or more polynucleotides encoding HBV antigens for use in treating an HBV infection in a subject in need thereof.

In one embodiment, the arenavirus vector comprises at least one of:
a) a first polynucleotide sequence encoding a truncated HBV core antigen consisting of an amino acid sequence that is at least 95%, 96%, 97%, 98%, 99% or 100% identical to SEQ ID NO: 2 or SEQ ID NO: 4, and
b) a second polynucleotide sequence encoding the HBV polymerase antigen consisting of an amino acid sequence that is at least 90%, such as at least 90%, 91%, 92%, 93%, 94%, 95%, 96%, 97%, 98%, 99% or 100% identical to SEQ ID NO: 7, wherein the HBV polymerase antigen does not have reverse transcriptase activity and RNase H activity,
   preferably, an arenavirus open reading frame is removed and replaced by the at least one of the first polynucleotide sequence and the second polynucleotide sequence, and the arenavirus vector is infectious

In one embodiment, the arenavirus vector comprises the first polynucleotide sequence encoding a truncated HBV core antigen consisting of an amino acid sequence that is at least 95% identical to SEQ ID NO: 2 or SEQ ID NO: 4. In another embodiment, the arenavirus vector comprises the second polynucleotide encoding the HBV polymerase antigen consisting of an amino acid sequence that is at least 90% identical to SEQ ID NO: 7.

In certain embodiments, the first polynucleotide sequence further comprises a polynucleotide sequence encoding a signal sequence operably linked to the N-terminus of the truncated HBV core antigen, and the second polynucleotide sequence further comprises a polynucleotide sequence encoding a signal sequence operably linked to the N-terminus of the HBV polymerase antigen, preferably, the signal sequence independently comprises the amino acid sequence of SEQ ID NO: 9 or SEQ ID NO: 15, preferably the signal sequence is independently encoded by the polynucleotide sequence of SEQ ID NO: 8 or SEQ ID NO: 14, respectively.

In certain embodiments, the first polynucleotide sequence encoding a truncated HBV core antigen consisting of an amino acid sequence of SEQ ID NO: 2 or SEQ ID NO: 4; and the second polynucleotide sequence encoding the HBV polymerase antigen consisting of an amino acid sequence of SEQ ID NO: 7. Preferably, the arenavirus vector comprises a) a first polynucleotide sequence encoding an truncated HBV core antigen consisting of the amino acid sequence of SEQ ID NO: 2 or SEQ ID NO: 4; and b) a second polynucleotide sequence encoding an HBV polymerase antigen having the amino acid sequence of SEQ ID NO: 7.

In certain embodiments, the first polynucleotide sequence comprises the polynucleotide sequence having at least 90%, such as at least 90%, 91%, 92%, 93%, 94%, 95%, 96%, 97%, 98%, 99% or 100%, sequence identity to SEQ ID NO: 1 or SEQ ID NO: 3.

In certain embodiments, the second polynucleotide sequence comprises a polynucleotide sequence having at least 90%, such as at least 90%, 91%, 92%, 93%, 94%, 95%, 96%, 97%, 98%, 99% or 100%, sequence identity to SEQ ID NO: 5 or SEQ ID NO: 6.

In an embodiment, the arenavirus vector encodes a fusion protein comprising the truncated the truncated HBV core antigen operably linked to the HBV polymerase antigen. In certain embodiments, the fusion protein comprises the truncated HBV core antigen operably linked to the HBV polymerase antigen via a linker. Preferably, the linker comprises the amino acid sequence of (AlaGly)n, and n is an integer of 2 to 5, preferably the linker is encoded by a polynucleotide sequence comprising SEQ ID NO: 11. Preferably, the fusion protein comprises the amino acid sequence of SEQ ID NO: 16.

In certain embodiments, examples of arenavirus vectors, compositions and methods to create and use such vectors for delivering genes of interest are described in U.S. Patent Application Publication US2018/0319845, International Patent Application Publication WO2017076988, the relevant content of each of which is hereby incorporated by reference in its entirety.

In certain embodiments, the arenavirus vector is infectious, i.e., it can enter into or inject its genetic material into a host cell. In certain embodiments, the infectious arenavirus viral vector is replication-deficient. In certain embodiments, the infectious arenavirus viral vector is replication-competent. In certain embodiments, the infectious, replication-deficient arenavirus viral vector is bisegmented. In certain embodiments, the infectious, replication-deficient arenavirus viral vector is trisegnrented. In certain embodiments, the infectious, replication-competent arenavirus viral vector is trisegmented.

In certain more specific embodiments, an arenavirus viral vector as provided herein can enter into or inject its genetic material into a host cell followed by amplification and expression of its genetic information inside the host cell. In certain embodiments, the viral vector is an infectious, replication-deficient arenavirus viral vector engineered to contain a genome with the ability to amplify and express its genetic information in infected cells but unable to produce further infectious progeny particles in normal, not genetically engineered cells that can support viral growth of a wild type virus but does not express the complementing viral protein, thus are tillable to produce further infectious viral progeny particles. In certain embodiments, the infectious arenavirus viral vector is replication-competent and able to produce further infectious progeny particles in normal, not genetically engineered cells.

In another general aspect, the application relates to a composition comprising an arenavirus vector of the application and a pharmaceutically acceptable carrier.

In certain embodiments, the composition comprises a first polynucleotide encoding a truncated HBV core antigen, a second polynucleotide sequence encoding the HBV polymerase antigen, and a pharmaceutically acceptable carrier, wherein the first and second polynucleotides are not comprised in the same arenavirus viral vector. In another embodiment, the first and second polynucleotides are comprised in the same arenavirus viral vector.

More preferably, the therapeutic composition comprises modified arenavirus particles in which an open reading frame of the arenavirus genome is deleted or functionally inactivated such that the resulting virus cannot produce further infectious progeny, but it transcribes at least one of the following polynucleotide sequences: a) a first polynucleotide sequence of SEQ ID NO: 1 or SEQ ID NO: 3; b) a second polynucleotide sequence of SEQ ID NO: 5 or 6.

The application further relates to a kit of the application for use in treating an HBV-induced disease in a subject in need thereof; and use kit of the application in the manufacture of a medicament for treating an HBV-induced disease in a subject in need thereof. The use can further comprise a combination with another therapeutic agent, preferably another anti-HBV antigen. Preferably, the subject has chronic HBV infection, and the HBV-induced disease is selected from the group consisting of advanced fibrosis, cirrhosis, and hepatocellular carcinoma (HCC).

The application also relates to a method of inducing an immune response against an HBV or a method of treating an HBV infection or an HBV-induced disease, comprising administering to a subject in need thereof an arenavirus vector or composition according to embodiments of the invention.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1A and FIG. 1B show schematic representations of DNA plasmids expressing a HBV gene according to embodiments of the application; FIG. 1A shows a DNA plasmid encoding an HBV core antigen according to an embodiment of the application; FIG. 1B shows a DNA plasmid encoding an HBV polymerase (pol) antigen according to an embodiment of the application; the HBV core and pol antigens are expressed under control of a CMV promoter with an N-terminal cystatin S signal peptide that is cleaved from the expressed antigen upon secretion from the cell; transcriptional regulatory elements of the plasmid include an enhancer sequence located between the CMV promoter and the polynucleotide sequence encoding the HBV antigen and a bGH polyadenylation sequence located downstream of the polynucleotide sequence encoding the HBV antigen; a second expression cassette is included in the plasmid in reverse orientation including a kanamycin resistance gene under control of an Ampr (bla) promoter; an origin of replication (pUC) is also included in reverse orientation.

FIG. 2A and FIG. 2B. show the schematic representations of the expression cassettes in adenoviral vectors according to embodiments of the application; FIG. 2A shows the expression cassette for a truncated HBV core antigen, which contains a CMV promoter, an intron (a fragment derived from the human ApoAI gene—GenBank accession X01038 base pairs 295-523, harboring the ApoAI second intron), a human immunoglobulin secretion signal, followed by a coding sequence for a truncated HBV core antigen and a SV40 polyadenylation signal; FIG. 2B shows the expression cassette for a fusion protein of a truncated HBV core antigen operably linked to an HBV polymerase antigen, which is otherwise identical to the expression cassette for the truncated HBV core antigen except the HBV antigen.

FIG. 3 shows ELISPOT responses of Balb/c mice immunized with different DNA plasmids expressing HBV core antigen or HBV pol antigen, as described in Example 3; peptide pools used to stimulate splenocytes isolated from the various vaccinated animal groups are indicated in gray scale; the number of responsive T-cells are indicated on the y-axis expressed as spot forming cells (SFC) per 10⁶ splenocytes.

FIG. 4. shows a schematic representation of the genome of wild type arenaviruses, which consists of a short (1; −3.4 kb) and a large (2; −7.2 kb) RNA segment. The short segment carries ORFs encoding the nucleoprotein (3) and glycoprotein (4). The large segment encodes the RNA-dependent RNA polymerase L (5) and the matrix protein Z (6). Wild type arenaviruses can be rendered replication-deficient vaccine vectors by deleting the glycoprotein gene and inserting, instead of the glycoprotein gene, antigens of choice (7) against which immune responses are to be induced (reproduced from FIG. 1 of US2018/0319845).

FIGS. 5A-5C: Schematic representation of the genomic organization of bi- and tri-segmented LCMV. The bi-segmented genome of wild-type LCMV consists of one S segment encoding the GP and NP and one L segment encoding the Z protein and the L protein (A). Both segments are flanked by the respective 5′ and 3′ UTRs. The genome of recombinant tri- segmented LCMVs (r3LCMV) consists of one L and two S segments with one position where to insert a gene of interest (here GFP) into each one of the S segments. r3LCMV-GFP^{natural} (nat) has all viral genes in their natural position (B), whereas the GP ORF in r3LCMV-GFP^{artificial} (art) is artificially juxtaposed to and expressed under control of the 3′ UTR (C) (reproduced from FIGS. 2A-2C of US2018/0319845).

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the application can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1 mg/mL to 10 mg/mL includes 0.9 mg/mL to 11 mg/mL. As used herein, the use of a numerical range expressly includes all possible subranges, all individual numerical values within that range, including integers within such ranges and fractions of the values unless the context clearly indicates otherwise.

The phrases “percent (%) sequence identity” or “% identity” or “% identical to” when used with reference to an amino acid sequence describe the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 91%, 92%, 93%, 94%, 95%, 97%, 98%, 99%, or 100% identity over the full-length of the amino acid sequences) can be determined, when the sequences are compared and aligned for maximum correspondence as measured using a sequence comparison algorithm as known in the art, or when manually aligned and visually inspected. The sequences which are compared to determine sequence identity can thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g. using the NCBI BLAST algorithm (Altschul SF, et al (1997), Nucleic Acids Res. 25:3389-3402).

As used herein, the terms and phrases “in combination,” “in combination with,” “co-delivery,” and “administered together with” in the context of the administration of two or more therapies or components to a subject refers to simultaneous administration or subsequent administration of two or more therapies or components, such as two vectors, e.g., RNA replicons, peptides, or a therapeutic combination and an adjuvant. “Simultaneous administration” can be administration of the two or more therapies or components at least within the same day. When two components are “administered together with” or “administered in combination with,” they can be administered in separate compositions sequentially within a short time period, such as 24, 20, 16, 12, 8 or 4 hours, or within 1 hour, or they can be administered in a single composition at the same time. “Subsequent administration” can be administration of the two or more therapies or components in the same day or on separate days. The use of the term “in combination with” does not restrict the order in which therapies or components are administered to a subject. For example, a first therapy or component (e.g. first arenavirus vector encoding an HBV antigen) can be administered prior to (e.g., 5 minutes to one hour before), concomitantly with or simultaneously with, or subsequent to (e.g., 5 minutes to one hour after) the administration of a second therapy or component (e.g., second arenavirus vector encoding an HBV antigen). In some embodiments, a first therapy or component (e.g. first arenavirus vector encoding an HBV antigen) and a second therapy or component (e.g., second arenavirus vector encoding an HBV antigen) are administered in the same composition. In other embodiments, a first therapy or component (e.g. first arenavirus vector encoding an HBV antigen) and a second therapy or component (e.g., second arenavirus vector encoding an HBV antigen) are administered in separate compositions, such as two separate compositions.

As used herein, a “non-naturally occurring” nucleic acid or polypeptide, refers to a nucleic acid or polypeptide that does not occur in nature. A “non-naturally occurring” nucleic acid or polypeptide can be synthesized, treated, fabricated, and/or otherwise manipulated in a laboratory and/or manufacturing setting. In some cases, a non-naturally occurring nucleic acid or polypeptide can comprise a naturally-occurring nucleic acid or polypeptide that is treated, processed, or manipulated to exhibit properties that were not present in the naturally-occurring nucleic acid or polypeptide, prior to treatment. As used herein, a “non-naturally occurring” nucleic acid or polypeptide can be a nucleic acid or polypeptide isolated or separated from the natural source in which it was discovered, and it lacks covalent bonds to sequences with which it was associated in the natural source. A “non-naturally occurring” nucleic acid or polypeptide can be made recombinantly or via other methods, such as chemical synthesis.

As used herein, “subject” means any animal, preferably a mammal, most preferably a human, to whom will be or has been treated by a method according to an embodiment of the application. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

As used herein, the term “operably linked” refers to a linkage or a juxtaposition wherein the components so described are in a relationship permitting them to function in their intended manner. For example, a regulatory sequence operably linked to a nucleic acid sequence of interest is capable of directing the transcription of the nucleic acid sequence of interest, or a signal sequence operably linked to an amino acid sequence of interest is capable of secreting or translocating the amino acid sequence of interest over a membrane.

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples. For example, while embodiments of HBV vectors of the application (e.g., arenavirus vectors) described herein can contain particular components, including, but not limited to, certain promoter sequences, enhancer or regulatory sequences, signal peptides, coding sequence of an HBV antigen, polyadenylation signal sequences, etc. arranged in a particular order, those having ordinary skill in the art will appreciate that the concepts disclosed herein can equally apply to other components arranged in other orders that can be used in HBV vectors of the application. The application contemplates use of any of the applicable components in any combination having any sequence that can be used in HBV vectors of the application, whether or not a particular combination is expressly described. The invention generally relates to an arenavirus vector encoding one or more HBV antigens.

### Hepatitis B Virus (HBV)

As used herein “hepatitis B virus” or “HBV” refers to a virus of the hepadnaviridae family. HBV is a small (e.g., 3.2 kb) hepatotropic DNA virus that encodes four open reading frames and seven proteins. The seven proteins encoded by HBV include small (S), medium (M), and large (L) surface antigen (HBsAg) or envelope (Env) proteins, pre-Core protein, core protein, viral polymerase (Pol), and HBx protein. HBV expresses three surface antigens, or envelope proteins, L, M, and S, with S being the smallest and L being the largest. The extra domains in the M and L proteins are named Pre-S2 and Pre-S1, respectively. Core protein is the subunit of the viral nucleocapsid. Pol is needed for synthesis of viral DNA (reverse transcriptase, RNaseH, and primer), which takes place in nucleocapsids localized to the cytoplasm of infected hepatocytes. PreCore is the core protein with an N-terminal signal peptide and is proteolytically processed at its N and C termini before secretion from infected cells, as the so-called hepatitis B e-antigen (HBeAg). HBx protein is required for efficient transcription of covalently closed circular DNA (cccDNA). HBx is not a viral structural protein. All viral proteins of HBV have their own mRNA except for core and polymerase, which share an mRNA. With the exception of the protein pre-Core, none of the HBV viral proteins are subject to post-translational proteolytic processing.

The HBV virion contains a viral envelope, nucleocapsid, and single copy of the partially double-stranded DNA genome. The nucleocapsid comprises 120 dimers of core protein and is covered by a capsid membrane embedded with the S, M, and L viral envelope or surface antigen proteins. After entry into the cell, the virus is uncoated and the capsid-containing relaxed circular DNA (rcDNA) with covalently bound viral polymerase migrates to the nucleus. During that process, phosphorylation of the core protein induces structural changes, exposing a nuclear localization signal enabling interaction of the capsid with so-called importins. These importins mediate binding of the core protein to nuclear pore complexes upon which the capsid disassembles and polymerase/rcDNA complex is released into the nucleus. Within the nucleus the rcDNA becomes deproteinized (removal of polymerase) and is converted by host DNA repair machinery to a covalently closed circular DNA (cccDNA) genome from which overlapping transcripts encode for HBeAg, HBsAg, Core protein, viral polymerase and HBx protein. Core protein, viral polymerase, and pre-genomic RNA (pgRNA) associate in the cytoplasm and self-assemble into immature pgRNA-containing capsid particles, which further convert into mature rcDNA-capsids and function as a common intermediate that is either enveloped and secreted as infectious virus particles or transported back to the nucleus to replenish and maintain a stable cccDNA pool.

To date, HBV is divided into four serotypes (adr, adw, ayr, ayw) based on antigenic epitopes present on the envelope proteins, and into eight genotypes (A, B, C, D, E, F, G, and H) based on the sequence of the viral genome. The HBV genotypes are distributed over different geographic regions. For example, the most prevalent genotypes in Asia are genotypes B and C. Genotype D is dominant in Africa, the Middle East, and India, whereas genotype A is widespread in Northern Europe, sub-Saharan Africa, and West Africa.

### HBV Antigens

As used herein, the terms “HBV antigen,” “antigenic polypeptide of HBV,” “HBV antigenic polypeptide,” “HBV antigenic protein,” “HBV immunogenic polypeptide,” and “HBV immunogen” all refer to a polypeptide capable of inducing an immune response, e.g., a humoral and/or cellular mediated response, against an HBV in a subject. The HBV antigen can be a polypeptide of HBV, a fragment or epitope thereof, or a combination of multiple HBV polypeptides, portions or derivatives thereof. An HBV antigen is capable of raising in a host a protective immune response, e.g., inducing an immune response against a viral disease or infection, and/or producing an immunity (i.e., vaccinates) in a subject against a viral disease or infection, that protects the subject against the viral disease or infection. For example, an HBV antigen can comprise a polypeptide or immunogenic fragment(s) thereof from any HBV protein, such as HBeAg, pre-core protein, HBsAg (S, M, or L proteins), core protein, viral polymerase, or HBx protein derived from any HBV genotype, e.g., genotype A, B, C, D, E, F, G, and/or H, or combination thereof.

### (1) HBV Core Antigen

As used herein, each of the terms “HBV core antigen,” “HBc” and “core antigen” refers to an HBV antigen capable of inducing an immune response, e.g., a humoral and/or cellular mediated response, against an HBV core protein in a subject. Each of the terms “core,” “core polypeptide,” and “core protein” refers to the HBV viral core protein. Full-length core antigen is typically 183 amino acids in length and includes an assembly domain (amino acids 1 to 149) and a nucleic acid binding domain (amino acids 150 to 183). The 34-residue nucleic acid binding domain is required for pre-genomic RNA encapsidation. This domain also functions as a nuclear import signal. It comprises 17 arginine residues and is highly basic, consistent with its function. HBV core protein is dimeric in solution, with the dimers self-assembling into icosahedral capsids. Each dimer of core protein has four α-helix bundles flanked by an α-helix domain on either side. Truncated HBV core proteins lacking the nucleic acid binding domain are also capable of forming capsids.

In an embodiment of the application, an HBV antigen is a truncated HBV core antigen. As used herein, a “truncated HBV core antigen,” refers to an HBV antigen that does not contain the entire length of an HBV core protein, but is capable of inducing an immune response against the HBV core protein in a subject. For example, an HBV core antigen can be modified to delete one or more amino acids of the highly positively charged (arginine rich) C-terminal nucleic acid binding domain of the core antigen, which typically contains seventeen arginine (R) residues. A truncated HBV core antigen of the application is preferably a C-terminally truncated HBV core protein which does not comprise the HBV core nuclear import signal and/or a truncated HBV core protein from which the C-terminal HBV core nuclear import signal has been deleted. In an embodiment, a truncated HBV core antigen comprises a deletion in the C-terminal nucleic acid binding domain, such as a deletion of 1 to 34 amino acid residues of the C-terminal nucleic acid binding domain, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acid residues, preferably a deletion of all 34 amino acid residues. In a preferred embodiment, a truncated HBV core antigen comprises a deletion in the C-terminal nucleic acid binding domain, preferably a deletion of all 34 amino acid residues.

An HBV core antigen of the application can be a consensus sequence derived from multiple HBV genotypes (e.g., genotypes A, B, C, D, E, F, G, and H). As used herein, “consensus sequence” means an artificial sequence of amino acids based on an alignment of amino acid sequences of homologous proteins, e.g., as determined by an alignment (e.g., using Clustal Omega) of amino acid sequences of homologous proteins. It can be the calculated order of most frequent amino acid residues, found at each position in a sequence alignment, based upon sequences of HBV antigens (e.g., core, pol, etc.) from at least 100 natural HBV isolates. A consensus sequence can be non-naturally occurring and different from the native viral sequences. Consensus sequences can be designed by aligning multiple HBV antigen sequences from different sources using a multiple sequence alignment tool, and at variable alignment positions, selecting the most frequent amino acid. Preferably, a consensus sequence of an HBV antigen is derived from HBV genotypes B, C, and D. The term “consensus antigen” is used to refer to an antigen having a consensus sequence.

An exemplary truncated HBV core antigen according to the application lacks the nucleic acid binding function, and is capable of inducing an immune response in a mammal against at least two HBV genotypes. Preferably a truncated HBV core antigen is capable of inducing a T cell response in a mammal against at least HBV genotypes B, C and D. More preferably, a truncated HBV core antigen is capable of inducing a CD8 T cell response in a human subject against at least HBV genotypes A, B, C and D.

Preferably, an HBV core antigen of the application is a consensus antigen, preferably a consensus antigen derived from HBV genotypes B, C, and D, more preferably a truncated consensus antigen derived from HBV genotypes B, C, and D. An exemplary truncated HBV core consensus antigen according to the application consists of an amino acid sequence that is at least 90% identical to SEQ ID NO: 2 or SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, or 100% identical to SEQ ID NO: 2 or SEQ ID NO: 4. SEQ ID NO: 2 and SEQ ID NO: 4 are core consensus antigens derived from HBV genotypes B, C, and D. SEQ ID NO: 2 and SEQ ID NO: 4 each contain a 34-amino acid C-terminal deletion of the highly positively charged (arginine rich) nucleic acid binding domain of the native core antigen.

In one embodiment of the application, an HBV core antigen is a truncated HBV antigen consisting of the amino acid sequence of SEQ ID NO: 2. In another embodiment, an HBV core antigen is a truncated HBV antigen consisting of the amino acid sequence of SEQ ID NO: 4. In another embodiment, an HBV core antigen further contains a signal sequence operably linked to the N-terminus of a mature HBV core antigen sequence, such as the amino acid sequence of SEQ ID NO: 2 or SEQ ID NO: 4. Preferably, the signal sequence has the amino acid sequence of SEQ ID NO: 9 or SEQ ID NO: 15.

### (2) HBV Polymerase Antigen

As used herein, the term “HBV polymerase antigen,” “HBV Pol antigen” or “HBV pol antigen” refers to an HBV antigen capable of inducing an immune response, e.g., a humoral and/or cellular mediated response, against an HBV polymerase in a subject. Each of the terms “polymerase,” “polymerase polypeptide,” “Pol” and “pol” refers to the HBV viral DNA polymerase. The HBV viral DNA polymerase has four domains, including, from the N terminus to the C terminus, a terminal protein (TP) domain, which acts as a primer for minus-strand DNA synthesis; a spacer that is nonessential for the polymerase functions; a reverse transcriptase (RT) domain for transcription; and a RNase H domain.

In an embodiment of the application, an HBV antigen comprises an HBV Pol antigen, or any immunogenic fragment or combination thereof. An HBV Pol antigen can contain further modifications to improve immunogenicity of the antigen, such as by introducing mutations into the active sites of the polymerase and/or RNase domains to decrease or substantially eliminate certain enzymatic activities.

Preferably, an HBV Pol antigen of the application does not have reverse transcriptase activity and RNase H activity and is capable of inducing an immune response in a mammal against at least two HBV genotypes. Preferably, an HBV Pol antigen is capable of inducing a T cell response in a mammal against at least HBV genotypes B, C and D. More preferably, an HBV Pol antigen is capable of inducing a CD8 T cell response in a human subject against at least HBV genotypes A, B, C and D.

Thus, in some embodiments, an HBV Pol antigen is an inactivated Pol antigen. In an embodiment, an inactivated HBV Pol antigen comprises one or more amino acid mutations in the active site of the polymerase domain. In another embodiment, an inactivated HBV Pol antigen comprises one or more amino acid mutations in the active site of the RNaseH domain. In a preferred embodiment, an inactivated HBV pol antigen comprises one or more amino acid mutations in the active site of both the polymerase domain and the RNaseH domain. For example, the “YXDD” motif in the polymerase domain of an HBV pol antigen that can be required for nucleotide/metal ion binding can be mutated, e.g., by replacing one or more of the aspartate residues (D) with asparagine residues (N), eliminating or reducing metal coordination function, thereby decreasing or substantially eliminating reverse transcriptase function. Alternatively, or in addition to mutation of the “YXDD” motif, the “DEDD” motif in the RNaseH domain of an HBV pol antigen required for Mg2+ coordination can be mutated, e.g., by replacing one or more aspartate residues (D) with asparagine residues (N) and/or replacing the glutamate residue (E) with glutamine (Q), thereby decreasing or substantially eliminating RNaseH function. In a particular embodiment, an HBV pol antigen is modified by (1) mutating the aspartate residues (D) to asparagine residues (N) in the “YXDD” motif of the polymerase domain; and (2) mutating the first aspartate residue (D) to an asparagine residue (N) and the glutamate residue (E) to a glutamine residue (N) in the “DEDD” motif of the RNaseH domain, thereby decreasing or substantially eliminating both the reverse transcriptase and RNaseH functions of the pol antigen.

In a preferred embodiment of the application, an HBV pol antigen is a consensus antigen, preferably a consensus antigen derived from HBV genotypes B, C, and D, more preferably an inactivated consensus antigen derived from HBV genotypes B, C, and D. An exemplary HBV pol consensus antigen according to the application comprises an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7, preferably at least 98% identical to SEQ ID NO: 7, such as at least 98%, 98.5%, 99%, 99.1%, 99.2%, 99.3%, 99.4%, 99.5%, 99.6%, 99.7%, 99.8%, 99.9% or 100% identical to SEQ ID NO: 7. SEQ ID NO: 7 is a pol consensus antigen derived from HBV genotypes B, C, and D comprising four mutations located in the active sites of the polymerase and RNaseH domains. In particular, the four mutations include mutation of the aspartic acid residues (D) to asparagine residues (N) in the “YXDD” motif of the polymerase domain; and mutation of the first aspartate residue (D) to an asparagine residue (N) and mutation of the glutamate residue (E) to a glutamine residue (Q) in the “DEDD” motif of the RNaseH domain.

In a particular embodiment of the application, an HBV pol antigen comprises the amino acid sequence of SEQ ID NO: 7. In other embodiments of the application, an HBV pol antigen consists of the amino acid sequence of SEQ ID NO: 7. In a further embodiment, an HBV pol antigen further contains a signal sequence operably linked to the N-terminus of a mature HBV pol antigen sequence, such as the amino acid sequence of SEQ ID NO: 7. Preferably, the signal sequence has the amino acid sequence of SEQ ID NO: 9 or SEQ ID NO: 15.

### (3) Fusion of HBV Core Antigen and HBV Polymerase Antigen

As used herein the term “fusion protein” or “fusion” refers to a single polypeptide chain having at least two polypeptide domains that are not normally present in a single, natural polypeptide.

In an embodiment of the application, an HBV antigen comprises a fusion protein comprising a truncated HBV core antigen operably linked to an HBV Pol antigen, or an HBV Pol antigen operably linked to a truncated HBV core antigen, preferably via a linker.

For example, in a fusion protein containing a first polypeptide and a second heterologous polypeptide, a linker serves primarily as a spacer between the first and second polypeptides. In an embodiment, a linker is made up of amino acids linked together by peptide bonds, preferably from 1 to 20 amino acids linked by peptide bonds, wherein the amino acids are selected from the 20 naturally occurring amino acids. In an embodiment, the 1 to 20 amino acids are selected from glycine, alanine, proline, asparagine, glutamine, and lysine. Preferably, a linker is made up of a majority of amino acids that are sterically unhindered, such as glycine and alanine. Exemplary linkers are polyglycines, particularly (Gly)5, (Gly)8; poly(Gly-Ala), and polyalanines. One exemplary suitable linker as shown in the Examples below is (AlaGly)n, wherein n is an integer of 2 to 5.

Preferably, a fusion protein of the application is capable of inducing an immune response in a mammal against HBV core and HBV Pol of at least two HBV genotypes. Preferably, a fusion protein is capable of inducing a T cell response in a mammal against at least HBV genotypes B, C and D. More preferably, the fusion protein is capable of inducing a CD8 T cell response in a human subject against at least HBV genotypes A, B, C and D.

In an embodiment of the application, a fusion protein comprises a truncated HBV core antigen having an amino acid sequence at least 90%, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, or 100% identical to SEQ ID NO: 2 or SEQ ID NO: 4, a linker, and an HBV Pol antigen having an amino acid sequence at least 90%, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, or 100%, identical to SEQ ID NO: 7.

In a preferred embodiment of the application, a fusion protein comprises a truncated HBV core antigen consisting of the amino acid sequence of SEQ ID NO: 2 or SEQ ID NO: 4, a linker comprising (AlaGly)n, wherein n is an integer of 2 to 5, and an HBV Pol antigen having the amino acid sequence of SEQ ID NO: 7. More preferably, a fusion protein according to an embodiment of the application comprises the amino acid sequence of SEQ ID NO: 16.

In one embodiment of the application, a fusion protein further comprises a signal sequence operably linked to the N-terminus of the fusion protein. Preferably, the signal sequence has the amino acid sequence of SEQ ID NO: 9 or SEQ ID NO: 15. In one embodiment, a fusion protein comprises the amino acid sequence of SEQ ID NO: 17.

Additional disclosure on HBV vaccines that can be used for the present invention are described in U.S. patent application Ser. No. 16/223,251, filed Dec. 18, 2018, the contents of the application, more preferably the examples, are hereby incorporated by reference in their entireties.

### Polynucleotides and Vectors

In another general aspect, the application provides a non-naturally occurring nucleic acid molecule encoding an HBV antigen useful for an invention according to embodiments of the application, and vectors comprising the non-naturally occurring nucleic acid. A first or second non-naturally occurring nucleic acid molecule can comprise any polynucleotide sequence encoding an HBV antigen useful for the application, which can be made using methods known in the art in view of the present disclosure. Preferably, a first or second polynucleotide encodes at least one of a truncated HBV core antigen and an HBV polymerase antigen of the application. A polynucleotide can be in the form of RNA or in the form of DNA obtained by recombinant techniques (e.g., cloning) or produced synthetically (e.g., chemical synthesis). The DNA can be single-stranded or double-stranded or can contain portions of both double-stranded and single-stranded sequence. The DNA can, for example, comprise genomic DNA, cDNA, or combinations thereof. The polynucleotide can also be a DNA/RNA hybrid. The polynucleotides and vectors of the application can be used for recombinant protein production, expression of the protein in host cell, or the production of viral particles. Preferably, a polynucleotide is RNA.

In an embodiment of the application, a first non-naturally occurring nucleic acid molecule comprises a first polynucleotide sequence encoding a truncated HBV core antigen consisting of an amino acid sequence that is at least 90% identical to SEQ ID NO: 2 or SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2 or SEQ ID NO: 4, preferably 98%, 99% or 100% identical to SEQ ID NO: 2 or SEQ ID NO: 4. In a particular embodiment of the application, a first non-naturally occurring nucleic acid molecule comprises a first polynucleotide sequence encoding a truncated HBV core antigen consisting the amino acid sequence of SEQ ID NO: 2 or SEQ ID NO: 4.

Examples of polynucleotide sequences of the application encoding a truncated HBV core antigen consisting of the amino acid sequence of SEQ ID NO: 2 or SEQ ID NO: 4 include, but are not limited to, a polynucleotide sequence at least 90% identical to SEQ ID NO: 1 or SEQ ID NO: 3, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 98%, 99% or 100% identical to SEQ ID NO: 1 or SEQ ID NO: 3. Exemplary non-naturally occurring nucleic acid molecules encoding a truncated HBV core antigen have the polynucleotide sequence of SEQ ID NOs: 1 or 3.

In another embodiment, a first non-naturally occurring nucleic acid molecule further comprises a coding sequence for a signal sequence that is operably linked to the N-terminus of the HBV core antigen sequence. Preferably, the signal sequence has the amino acid sequence of SEQ ID NO: 9 or SEQ ID NO: 15. More preferably, the coding sequence for a signal sequence comprises the polynucleotide sequence of SEQ ID NO: 8 or SEQ ID NO: 14.

In an embodiment of the application, a second non-naturally occurring nucleic acid molecule comprises a second polynucleotide sequence encoding an HBV polymerase antigen comprising an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7, preferably 100% identical to SEQ ID NO: 7. In a particular embodiment of the application, a second non-naturally occurring nucleic acid molecule comprises a second polynucleotide sequence encoding an HBV polymerase antigen consisting of the amino acid sequence of SEQ ID NO: 7.

Examples of polynucleotide sequences of the application encoding an HBV Pol antigen comprising the amino acid sequence of at least 90% identical to SEQ ID NO: 7 include, but are not limited to, a polynucleotide sequence at least 90% identical to SEQ ID NO: 5 or SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or SEQ ID NO: 6, preferably 98%, 99% or 100% identical to SEQ ID NO: 5 or SEQ ID NO: 6. Exemplary non-naturally occurring nucleic acid molecules encoding an HBV pol antigen have the polynucleotide sequence of SEQ ID NOs: 5 or 6.

In another embodiment, a second non-naturally occurring nucleic acid molecule further comprises a coding sequence for a signal sequence that is operably linked to the N-terminus of the HBV pol antigen sequence, such as the amino acid sequence of SEQ ID NO: 7. Preferably, the signal sequence has the amino acid sequence of SEQ ID NO: 9 or SEQ ID NO: 15. More preferably, the coding sequence for a signal sequence comprises the polynucleotide sequence of SEQ ID NO: 8 or SEQ ID NO: 14.

In another embodiment of the application, a non-naturally occurring nucleic acid molecule encodes an HBV antigen fusion protein comprising a truncated HBV core antigen operably linked to an HBV Pol antigen, or an HBV Pol antigen operably linked to a truncated HBV core antigen. In a particular embodiment, a non-naturally occurring nucleic acid molecule of the application encodes a truncated HBV core antigen consisting of an amino acid sequence that is at least 90% identical to SEQ ID NO: 2 or SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2 or SEQ ID NO: 4, preferably 100% identical to SEQ ID NO: 2 or SEQ ID NO: 4, more preferably 100% identical to SEQ ID NO: 2 or SEQ ID NO:4; a linker; and an HBV polymerase antigen comprising an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7, preferably 98%, 99% or 100% identical to SEQ ID NO: 7. In a particular embodiment of the application, a non-naturally occurring nucleic acid molecule encodes a fusion protein comprising a truncated HBV core antigen consisting of the amino acid sequence of SEQ ID NO: 2 or SEQ ID NO: 4, a linker comprising (AlaGly)n, wherein n is an integer of 2 to 5; and an HBV Pol antigen comprising the amino acid sequence of SEQ ID NO: 7. In a particular embodiment of the application, a non-naturally occurring nucleic acid molecule encodes an HBV antigen fusion protein comprising the amino acid sequence of SEQ ID NO: 16.

Examples of polynucleotide sequences of the application encoding an HBV antigen fusion protein include, but are not limited to, a polynucleotide sequence at least 90% identical to SEQ ID NO: 1 or SEQ ID NO: 3, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 98%, 99% or 100% identical to SEQ ID NO: 1 or SEQ ID NO: 3, operably linked to a linker coding sequence at least 90% identical to SEQ ID NO: 11, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 11, preferably 98%, 99% or 100% identical to SEQ ID NO: 11, which is further operably linked a polynucleotide sequence at least 90% identical to SEQ ID NO: 5 or SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or SEQ ID NO: 6, preferably 98%, 99% or 100% identical to SEQ ID NO: 5 or SEQ ID NO: 6. In particular embodiments of the application, a non-naturally occurring nucleic acid molecule encoding an HBV antigen fusion protein comprises SEQ ID NO: 1 or SEQ ID NO: 3, operably linked to SEQ ID NO: 11, which is further operably linked to SEQ ID NO: 5 or SEQ ID NO: 6.

In another embodiment, a non-naturally occurring nucleic acid molecule encoding an HBV fusion further comprises a coding sequence for a signal sequence that is operably linked to the N-terminus of the HBV fusion sequence, such as the amino acid sequence of SEQ ID NO: 16. Preferably, the signal sequence has the amino acid sequence of SEQ ID NO: 9 or SEQ ID NO: 15. More preferably, the coding sequence for a signal sequence comprises the polynucleotide sequence of SEQ ID NO: 8 or SEQ ID NO: 14. In one embodiment, the encoded fusion protein with the signal sequence comprises the amino acid sequence of SEQ ID NO: 17.

The application also relates to a vector comprising the first and/or second non-naturally occurring nucleic acid molecules. As used herein, a “vector” is a nucleic acid molecule used to carry genetic material into another cell, where it can be replicated and/or expressed. A vector of the application can be an expression vector. As used herein, the term “expression vector” refers to any type of genetic construct comprising a nucleic acid coding for an RNA capable of being transcribed. Expression vectors include, but are not limited to, vectors for recombinant protein expression, such as an RNA replicon or a viral vector, and vectors for delivery of nucleic acid into a subject for expression in a tissue of the subject, such as an RNA replicon or a viral vector. It will be appreciated by those skilled in the art that the design of the expression vector can depend on such factors as the choice of the host cell to be transformed, the level of expression of protein desired, etc.

A vector can comprise one or more expression cassettes. An “expression cassette” is part of a vector that directs the cellular machinery to make RNA and protein. An expression cassette typically comprises three components: a promoter sequence, an open reading frame, and a 3′-untranslated region (UTR) optionally comprising a polyadenylation signal. An open reading frame (ORF) is a reading frame that contains a coding sequence of a protein of interest (e.g., HBV antigen) from a start codon to a stop codon. Regulatory elements of the expression cassette can be operably linked to a polynucleotide sequence encoding an HBV antigen of interest. As used herein, the term “operably linked” is to be taken in its broadest reasonable context and refers to a linkage of polynucleotide elements in a functional relationship. A polynucleotide is “operably linked” when it is placed into a functional relationship with another polynucleotide. For instance, a promoter is operably linked to a coding sequence if it affects the transcription of the coding sequence. Any components suitable for use in an expression cassette described herein can be used in any combination and in any order to prepare vectors of the application.

A vector can comprise a promoter sequence, preferably within an expression cassette, to control expression of an HBV antigen of interest. The term “promoter” is used in its conventional sense and refers to a nucleotide sequence that initiates the transcription of an operably linked nucleotide sequence. A promoter is located on the same strand near the nucleotide sequence it transcribes. Promoters can be a constitutive, inducible, or repressible. Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). Preferably, the promoter is located upstream of the polynucleotide encoding an HBV antigen within an expression cassette.

Examples of promoters that can be used include, but are not limited to, a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. A promoter can also be a promoter from a human gene such as human actin, human myosin, human hemoglobin, human muscle creatine, or human metalothionein. A promoter can also be a tissue specific promoter, such as a muscle or skin specific promoter, natural or synthetic.

For example, a promoter can be a strong eukaryotic promoter, such as a cytomegalovirus immediate early (CMV-IE) promoter. A nucleotide sequence of an exemplary CMV-IE promoter is shown in SEQ ID NO: 18 or SEQ ID NO: 19.

A vector can comprise additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for a protein of interest (e.g., an HBV antigen) within an expression cassette of the vector. Enhancer sequences are regulatory DNA sequences that, when bound by transcription factors, enhance the transcription of an associated gene. An enhancer sequence is preferably located upstream of the polynucleotide sequence encoding an HBV antigen, but downstream of a promoter sequence within an expression cassette of the vector.

Any polyadenylation signal known to those skilled in the art in view of the present disclosure can be used. For example, the polyadenylation signal can be a SV40 polyadenylation signal, LTR polyadenylation signal, bovine growth hormone (bGH) polyadenylation signal, human growth hormone (hGH) polyadenylation signal, or human β-globin polyadenylation signal. Preferably, a polyadenylation signal is a bovine growth hormone (bGH) polyadenylation signal or a SV40 polyadenylation signal. A nucleotide sequence of an exemplary bGH polyadenylation signal is shown in SEQ ID NO: 20. A nucleotide sequence of an exemplary SV40 polyadenylation signal is shown in SEQ ID NO: 13.

Any enhancer sequence known to those skilled in the art in view of the present disclosure can be used. For example, an enhancer sequence can be human actin, human myosin, human hemoglobin, human muscle creatine, or a viral enhancer, such as one from CMV, HA, RSV, or EBV. Examples of particular enhancers include, but are not limited to, Woodchuck HBV Post-transcriptional regulatory element (WPRE), intron/exon sequence derived from human apolipoprotein A1 precursor (ApoAI), untranslated R-U5 domain of the human T-cell leukemia virus type 1 (HTLV-1) long terminal repeat (LTR), a splicing enhancer, a synthetic rabbit β-globin intron, or any combination thereof. Preferably, an enhancer sequence is a composite sequence of three consecutive elements of the untranslated R-U5 domain of HTLV-1 LTR, rabbit β-globin intron, and a splicing enhancer, which is referred to herein as “a triple enhancer sequence.” A nucleotide sequence of an exemplary triple enhancer sequence is shown in SEQ ID NO: 10. Another exemplary enhancer sequence is an ApoAI gene fragment shown in SEQ ID NO: 12.

A vector can comprise a polynucleotide sequence encoding a signal peptide sequence. Preferably, the polynucleotide sequence encoding the signal peptide sequence is located upstream of the polynucleotide sequence encoding an HBV antigen. Signal peptides typically direct localization of a protein, facilitate secretion of the protein from the cell in which it is produced, and/or improve antigen expression and cross-presentation to antigen-presenting cells. A signal peptide can be present at the N-terminus of an HBV antigen when expressed from the vector, but is cleaved off by signal peptidase, e.g., upon secretion from the cell. An expressed protein in which a signal peptide has been cleaved is often referred to as the “mature protein.” Any signal peptide known in the art in view of the present disclosure can be used. For example, a signal peptide can be a cystatin S signal peptide; an immunoglobulin (Ig) secretion signal, such as the Ig heavy chain gamma signal peptide SPIgG or the Ig heavy chain epsilon signal peptide SPIgE.

Preferably, a signal peptide sequence is a cystatin S signal peptide. Exemplary nucleic acid and amino acid sequences of a cystatin S signal peptide are shown in SEQ ID NOs: 8 and 9, respectively. Exemplary nucleic acid and amino acid sequences of an immunoglobulin secretion signal are shown in SEQ ID NOs: 14 and 15, respectively.

In one general aspect, provided herein is an arenavirus vector comprising the first and/or second non-naturally occurring nucleic acid molecules. In certain embodiments, the arenavirus vector is a genetically modified arenavirus, where the arenavirus is infectious, expresses its genetic information, and encodes an HBV antigen or a fragment thereof, but cannot form infectious progeny virus in a non-complementary cell (i.e., a cell that does not express the functionality that is missing from the replication-deficient arenavirus and causes it to be replication-deficient). An arenavirus vector of the application can be infectious, i.e., it can attach to a host cell and release its genetic material into the host cell. An arenavirus vector of the application can be replication-deficient, i.e., the arenavirus is unable to produce further infectious progeny particles in a non-complementing cell. To create a replication-deficient arenavirus, the genome of the arenavirus is modified (e.g., by deletion or functional inactivation of an ORF) such that a virus carrying the modified genome can no longer produce infectious progeny viruses. A non-complementing cell is a cell that does not provide the functionality that has been eliminated from the replication-deficient arenavirus by modification of the virus genome (e.g., if the ORF encoding the GP protein is deleted or functionally inactivated, a non-complementing cell does not provide the GP protein). However, a genetically modified replication-deficient arenavirus can produce infectious progeny viruses in complementing cells. Complementing cells are cells that provide (in trans) the functionality that has been eliminated from the replication-deficient arenavirus by modification of the virus genome (e.g., if the ORF encoding the GP protein is deleted or functionally inactivated, a complementing cell does provide the GP protein). Expression of the complementing functionality (e.g., the GP protein) can be accomplished by any method known to the skilled artisan (e.g., transient or stable expression). A genetically modified arenavirus described herein can amplify and express its genetic information in a cell that has been infected by the virus. A genetically modified arenavirus provided herein comprises a nucleotide sequence that encodes an HBV antigen such as, but not limited to, the HBV antigens described herein.

Arenaviruses for use with the methods and compositions provided herein can be Old World viruses, for example Lassa virus, Lymphocytic choriomeningitis virus (LCMV), Mobala virus, Mopeia virus, or Ippy virus, or New World viruses, for example Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Bear Canyon virus, or Whitewater Arroyo virus.

In certain embodiments, the vector generated to encode one or more HBV antigens can be based on a specific strain of LCMV. Strains of LCMV include Clone 13, MP strain, Arm CA 1371, Arm E-250, WE, UBC, Traub, Pasteur, 810885, CH-5692, Marseille #12, HP65-2009, 200501927, 810362, 811316, 810316, 810366, 20112714, Douglas, GRO1, SN05, CABN and their derivatives. In certain embodiments, the vector generated to encode one or more HBV antigens can be based on LCMV Clone 13. In other embodiments, the vector generated to encode one or more HBV antigens can be based on LCMV MP strain.

In certain embodiments, the vector generated to encode one or more HBV antigens can be based on a specific strain of Junin virus. Strains of Junin virus include vaccine strains XJ13, XJ #44, and Candid #1 as well as IV 4454, a human isolate. In certain embodiments, the vector generated to encode one or more HBV antigens is based on Junin virus Candid #1 strain.

The wild type arenavirus genome consists of a short (−3 .4 kb) and a large (−7 .2 kb) RNA segment (FIG. 4). The short segment carries the ORFs encoding the nucleoprotein NP and glycoprotein GP genes. The large segment com-prises the RNA-dependent RNA polymerase L and the matrix protein Z genes. The wild type arenavirus vector genome can be modified to retain at least the essential regulatory dements on the 5′ and 3′ untranslated regions UTRs) of both segments, and/or also the intergenic regions (IGRs). For example, the S segment of the arenavirus can be modified by substituting the ORF encoding the GP protein with an ORF encoding an HBV antigen.

Arenavirus disease and immunosuppression in wild type arenavirus infection are known to result from unchecked viral replication. By abolishing replication, i.e., the ability to produce infectious progeny virus particles, of arenavirus vectors by deleting from their genome, e.g., the Z gene which is required for particle release, or the GP gene which is required for infection of target cells, the total number of infected cells can be limited by the inoculum administered, e.g., to a vaccine recipient, or accidentally transmitted to personnel involved in medical or biotechno-logical applications, or to animals. Therefore, abolishing replication of arenavirus vectors prevents pathogenesis as a result of intentional or accidental transmission of vector particles. Provided herein, one important aspect consists in exploiting the above necessity of abolishment of replication in a beneficial way for the purpose of expressing an HBV antigen. In certain embodiments, an arenavirus particle is rendered replication deficient by genetic modification of its genome. Such modifications to the genome can include:
deletion of an ORF (e.g., the ORF encoding the GP, NP, L, or Z protein);
functional inactivation of an ORF (e.g., the ORF encoding the GP, NP, L, or Z protein), e.g., this can be achieved by introducing a missense or a nonsense muta-tion;
change of the sequence of the ORF (e.g., the exchange of an Sl 13 cleavage site with the cleavage site of another protease);
mutagenesis of one of the 5′ or 3′ termini of one of the genomic segments; mutagenesis of an intergenic region (i.e., of the L or the S genomic segment).

In certain embodiments, provided herein is an infectious arenavirus viral vector, wherein an arenavirus open reading frame is removed and replaced by a nucleotide sequence encoding a fusion of HBV core and polymerase proteins or antigenic fragments thereof. In specific embodiments, the arenavirus is lymphocytic choriomeningitis virus. In specific embodiments, the open reading frame that encodes the glycoprotein of the arenavirus is deleted or functionally inactivated. In specific embodiments, the viral vector is replication-deficient. In specific embodiments, the viral vector is replication-competent. In specific embodiments, the viral vector is tri-segmented. In certain embodiments, provided herein is a method of treating or preventing a Hepatitis B virus infection in a patient, wherein said method comprises administering to the patient the viral vector from which an arenavirus open reading frame is removed and replaced by a nucleotide sequence encoding a fusion of HBV core and polymerase proteins or antigenic fragments thereof.

Without being bound by theory, the minimal transacting factors for gene expression in infected cells remain in the vector genome as ORB that can be expressed, yet they can be placed differently in the genome and can be placed under control of a different promoter than naturally or can be expressed from internal ribosome entry sites. In certain embodiments, the nucleic acid encoding an HBV antigen is transcribed from one of the endogenous arenavirus promoters (i.e., 5′ UER, 3′ UTR of the S segment, 5′ UTR, 3′ UTR of the L segment). In other embodiments, the nucleic acid encoding an HBV antigen is expressed from a heterologous introduced promoter sequences that can be read by the viral RNA-dependent RNA polymerase, by cellular RNA polymerase RNA polymerase II or RNA polymerase III, such as duplications of viral promoter sequences that are naturally found in the viral UTRs, the 28S ribosomal RNA promoter, the beta-actin promoter or the 5S ribosomal RNA promoter, respectively. In certain embodiments ribonucleic acids coding for BEV antigens are transcribed and translated either by themselves or as read-through by fusion to arenavirus protein ORFS, and expression of proteins in the host cell may be enhanced by introducing in the viral transcript sequence at the appropriate place(s) one or more, e.g., two, three or four, internal ribosome entry sites.

In certain embodiments, an infectious arenavirus expressing an HBV antigen for use with the compositions and methods described herein is engineered to carry a viral ORF in a position other than the wild-type position of the ORF. In some embodiments, the arenavirus genomic segment is selected from the group consisting of: (i) an S segment, wherein the ORF encoding the NP is under control of an arenavirus 5′ UTR; (ii) an S segment, wherein the ORF encoding the Z protein is under control of an arenavirus 5′ UTR; (iii) an S segment, wherein the ORF encoding the L protein is under control of an arenavirus 5′ UTR; (iv) an S segment, wherein the ORF encoding the GP is under control of an arenavirus 3′ UTR; (v) an S segment, wherein the ORF encoding the L protein is under control of an arenavirus 3′ UTR; (vi) an S segment, wherein the ORF encoding the

Z protein is under control of an arenavirus 3′ UTR; (vii) an L segment, wherein the ORF encoding the GP is under control of an arenavirus 5′ UTR; (viii) an L segment, wherein the ORF encoding the NP is under control of an arenavirus 5′ UTR; (ix) an L segment, wherein the ORF encoding the L protein is under control of an arenavirus 5′ UTR; (x) an L segment, wherein the ORF encoding the GP is under control of an arenavirus 3′ UTR; (xi) an L segment, wherein the ORF encoding the NP is under control of an arenavirus 3′ UTR; and (xii) an L segment, wherein the ORF encoding the Z protein is under control of an arenavirus 3′ UTR.

In certain embodiments, the ORF encoding GP, NP, Z protein, or the L protein of the tri-segmented arenavirus particle described herein can be under the control of an arenavirus 3′ UTR or an arenavirus 5′ UTR. In more specific embodiments, the tri-segmented arenavirus 3′ UTR is the 3′ UTR of an arenavirus S segment(s). In another specific embodiment, the tri-segmented arenavirus 3′ UTR is the 3′ UTR of an arenavirus L segment(s). In more specific embodiments, the tri-segmented arenavirus 5′ UTR is the 5′ UTR of an arenavirus S segment(s). In other specific embodiments, the 5′ UTR is the 5′ UTR of an arenavirus L segment(s).

In other embodiments, the ORF encoding GP, NP, Z protein, or the L protein of a tri-segmented arenavirus particle described herein can be under the control of the arenavirus conserved terminal sequence element (the 5′- and 3′-terminal 19-20-nt regions) (see e.g., Perez & de la Torre, 2003, J Viral. 77(2): 1184-1194).

In certain embodiments, the ORF encoding GP, NP, Z protein or the L protein of the tri-segmented arenavirus particle can be under the control of the promoter element of the 5′ UTR (see e.g., Albarino et al., 2011, J Viral., 85(8): 4020-4). In another embodiment, the ORF encoding GP, NP Z protein, L protein of the tri-segmented arenavirus particle can be under the control of the promoter element of the 3′ UTR (see e.g., Albarino et al., 2011, J Viral., 85(8):4020-4). In more specific embodiments, the promoter element of the 5′ UTR is the 5′ UTR promoter element of the S segment(s) or the L segment(s). In another specific embodiment, the promoter element of the 3′ UTR is the 3′ UTR the promoter element of the S segment(s) or the L segment(s).

In certain embodiments, the ORF that encoding GP, NP, Z protein or the L protein of the tri-segmented arenavirus particle can be under the control of a truncated arenavirus 3′ UTR or a truncated arenavirus 5′ UTR (see e.g., Perez & de la Torre, 2003, J Viral. 77(2): 1184-1194; Albarino et al., 2011, J Viral., 85(8):4020-4). In more specific embodiments, the truncated 3′ UTR is the 3′ UTR of the arenavirus S segment or L segment. In more specific embodiments, the truncated 5′ UTR is the 5′ UTR of the arenavirus S segment(s) or L segment(s).

In certain embodiments, the ORF that encodes the glycoprotein of the arenavirus is substituted by a nucleic acid sequence encoding one or more HBV antigens described herein.

In some embodiments, the arenavirus 3′ UTR is the 3′ UTR of the arenavirus S segment or the arenavirus L segment. In certain embodiments, the arenavirus 5′ UTR is the 5′ UTR of the arenavirus S segment or the arenavirus L segment.

In certain embodiments, for use with the compositions and methods provided herein is a tri-segmented arenavirus particle comprising one L segment and two S segments in which (i) an ORF is in a position other than the wild-type position of the ORF; and (ii) an ORF encoding OP or NP has been removed or functionally inactivated, such that the resulting virus cannot produce further infectious progeny virus particles. In a specific embodiment, one ORF is removed and replaced with a heterologous ORF encoding an HBV antigen) from an organism other than an arenavirus. In another specific embodiment, two ORFS are removed and replaced with a heterologous ORF from an organism other than an arenavirus. In other specific embodiments, three ORFs are removed and replaced with a heterologous ORF (e.g., encoding an HBV antigen) from an organism other than an arenavirus. In specific embodiments, the ORF encoding GP is removed and replaced with a heterologous ORF (e.g., encoding an HBV antigen) from an organism other than an arenavirus. In other specific embodiments, the ORF encoding NP is removed and replaced with a heterologous ORF (e.g., encoding an HBV antigen) from an organism other than an arenavirus. In yet more specific embodiments, the ORF encoding NP and the ORF encoding GP are removed and replaced with one or two heterologous ORFs (e.g., encoding one or two HBV antigens) from an organism other than an arenavirus particle. Thus, in certain embodiments the tri-segmented arenavirus particle comprises (i) one L segment and two S segments; (ii) an ORF in a position other than the wild-type position of the ORF; (iii) one or more heterologous ORF (e.g., encoding one or more HBV antigens) from an organism other than an arenavirus.

In certain embodiments, for use with the compositions and methods provided herein is a tri-segmented arenavirus particle comprising two L segments and one S segment in which (i) an ORF is in a position other than the wild-type position of the ORF; and (ii) an ORF encoding the Z protein, and/or the L protein has been removed or functionally inactivated, such that the resulting virus cannot produce further infectious progeny virus particle. In a specific embodiment, one ORF is removed and replaced with a heterologous ORF (e.g., encoding an HBV antigen) from an organism other than an arenavirus. In another specific embodiment, two ORFs are removed and replaced with a heterologous ORF (e.g., encoding an HBV antigen) from an organism other than an arenavirus. In specific embodiments, the ORF encoding the Z protein is removed and replaced with a heterologous ORF (e.g., encoding an HBV antigen) from an organism other than an arenavirus. In other specific embodiments, the ORF encoding the L protein is removed and replaced with a heterologous ORF (e.g., encoding an HBV antigen) from an organism other than an arenavirus. In yet more specific embodiments, the ORF encoding the Z protein and the ORF encoding the L protein is removed and replaced with a heterologous ORF (e.g., encoding an HBV antigen) from an organism other than an arenavirus particle. Thus, in certain embodiments the tri-segmented arenavirus particle comprises (i) two L segments and one S segment; (ii) an ORF in a position other than the wild-type position of the ORF; (iii) heterologous ORF (e.g., encoding an HBV antigen) from an organism other than an arenavirus.

Thus, in certain embodiments, the tri-segmented arenavirus particle for use with the compositions and methods provided herein comprises a tri-segmented arenavirus particle (i.e., one L segment and two S segments or two L segments and one S segment) that i) is engineered to carry an ORF in a non-natural position; ii) an ORF encoding GP, NP, Z protein, or L protein is removed; and iii) the ORF that is removed is replaced with one or more heterologous ORFs (e.g., encoding one or more HBV antigens) from an organism other than an arenavirus.

In certain embodiments, the infectious arenavirus viral vector is replication-deficient. In certain embodiments, the infectious arenavirus viral vector is replication-competent.

In certain embodiments, the arenavirus vector is a replication-deficient, bisegmented arenavirus vector. In certain embodiments, the arenavirus vector is a replication-deficient, trisegmented arenavirus vector. Wild type arena-viruses can be rendered replication-deficient to generate vaccine vectors by substituting the glycoprotein gene for one or more HBV antigens, against which immune responses are to be induced.

In certain embodiments, an infectious arenavirus expressing an HBV antigen described herein is a Lympho-cytic choriomeningitis virus (LCMV) wherein the S segment of the virus is modified by substituting the ORF encoding the GP protein with an ORF encoding an HBV antigen.

In certain embodiments, the genomic information encoding the infectious arenavirus particle is derived from the LCMV Clone 13 strain or the LCMV MP strain. The nucleotide sequence of the S segment and of the L segment of Clone 13 are set forth in SEQ ID NOs: 25 and 26, respectively.

In certain embodiments, provided herein is a viral vector whose genome is or has been derived from the genome of Clone 13 by deleting an ORF of the Clone 13 genome (e.g., the ORF of the GP protein) and replacing it with a heterologous ORF that encodes an antigen (e.g., an HBV antigen) such that the remaining LCMV genome is at least 80%, 81%, 82%, 83%, 84%, 85%, 86%, 87%, 88%, 89%, 90%, 91%, 92%, 93%, 94%, 95%, 96%, 97%, 98%, at least 99%, or 100% identical to the nucleotide sequence of the unmodified Clone 13.

In certain embodiments, provided herein is a viral vector whose genome has been derived from the genome of the LCMV strain MP (SEQ ID NOs: 27 and 28 for the L segment and of the S segment, respectively) by deleting an ORF of the LCMV strain MP genome (e.g., the ORF of the GP protein) and replacing it with a heterologous ORF that encodes an antigen (e.g., an HBV antigen) such that the remaining LCMV genome is at least 80%, 81%, 82%, 83%, 84%, 85%, 86%, 87%, 88%, 89%, 90%, 91%, 92%, 93%, 94%, 95%, 96%, 97%, 98%, at least 99%, 99.1%, 99.2%, 99.3%, 99.4%, 99.5%, 99.6%, 99.7%, 99.8%, at least 99.9% or 100% identical to the nucleotide sequence of the unmodified LCMV strain MP.

In a more specific embodiment, the viral vector comprises a genomic segment, wherein the genomic segment comprises a nucleotide sequence that is at least 80%, 81%, 82%, 83%, 84%, 85%, 86%, 87%, 88%, 89%, 90%, 91%, 92%, 93%, 94%, 95%, 96%, 97%, 98%, at least 99%, or 100% identical to the sequence of nucleotide 1639 to 3315 of SEQID NO: 29 or 1640 to 3316 of SEQID NO: 25. In certain embodiments, the viral vector comprises a genomic segment comprising a nucleotide sequence encoding an expression product whose amino acid sequence is at least 80%, 81%, 82%, 83%, 84%, 85%, 86%, 87%, 88%, 89%, 90%, 91%, 92%, 93%, 94%, 95%, 96%, 97%, 98%, at least 99%, or 100% identical to the amino acid sequence encoded by 1639 to 3315 of SEQ ID NO: 29 or 1640 to 3316 of SEQ ID NO: 25.

In certain embodiments, the arenavirus is lymphocytic choriomeningitis virus (LCMV) or Junin virus (JUNV).

In a particular embodiment of the application, an arenavirus vector comprises an expression cassette including a polynucleotide encoding at least one of an HBV antigen selected from the group consisting of an HBV pol antigen comprising an amino acid sequence at least 90%, such as 90%, 91%, 92%, 93%, 94%, 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical to SEQ ID NO: 7, and a truncated HBV core antigen consisting of the amino acid sequence at least 95%, such as 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical of SEQ ID NO: 2 or SEQ ID NO: 4; an upstream sequence operably linked to the polynucleotide encoding the HBV antigen comprising, from 5′ end to 3′ end, a promoter sequence, preferably a CMV promoter sequence of SEQ ID NO: 18, an enhancer sequence, preferably a triple enhancer sequence of SEQ ID NO: 10, and a polynucleotide sequence encoding a signal peptide sequence, preferably a cystatin S signal peptide having the amino acid sequence of SEQ ID NO: 9; and a downstream sequence operably linked to the polynucleotide encoding the HBV antigen comprising a polyadenylation signal, preferably a bGH polyadenylation signal of SEQ ID NO: 20.

In another particular embodiment of the application, an arenavirus vector comprises an expression cassette including a polynucleotide encoding at least one of an HBV antigen selected from the group consisting of an HBV pol antigen comprising an amino acid sequence at least 90%, such as 90%, 91%, 92%, 93%, 94%, 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical to SEQ ID NO: 7, and a truncated HBV core antigen consisting of the amino acid sequence at least 95%, such as 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical of SEQ ID NO: 2 or SEQ ID NO: 4; an upstream sequence operably linked to the polynucleotide encoding the HBV antigen comprising, from 5′ end to 3′ end, a promoter sequence, preferably a CMV promoter sequence of SEQ ID NO: 19, an enhancer sequence, preferably an ApoAI gene fragment sequence of SEQ ID NO: 12, and a polynucleotide sequence encoding a signal peptide sequence, preferably an immunoglobulin secretion signal having the amino acid sequence of SEQ ID NO: 15; and a downstream sequence operably linked to the polynucleotide encoding the HBV antigen comprising a polyadenylation signal, preferably a SV40 polyadenylation signal of SEQ ID NO: 13.

In an embodiment of the application, an arenavirus vector encodes an HBV Pol antigen having the amino acid sequence of SEQ ID NO: 7. Preferably, the arenavirus vector comprises a coding sequence for the HBV Pol antigen that is at least 90% identical to the polynucleotide sequence of SEQ ID NO: 5 or 6, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or 6, preferably 100% identical to SEQ ID NO: 5 or 6.

In an embodiment of the application, an arenavirus vector encodes a truncated HBV core antigen consisting of the amino acid sequence of SEQ ID NO: 2 or SEQ ID NO: 4. Preferably, the arenavirus vector comprises a coding sequence for the truncated HBV core antigen that is at least 90% identical to the polynucleotide sequence of SEQ ID NO: 1 or SEQ ID NO: 3, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 100% identical to SEQ ID NO: 1 or SEQ ID NO: 3.

In yet another embodiment of the application, an arenavirus vector encodes a fusion protein comprising an HBV Pol antigen having the amino acid sequence of SEQ ID NO: 7 and a truncated HBV core antigen consisting of the amino acid sequence of SEQ ID NO: 2 or SEQ ID NO: 4. Preferably, the arenavirus vector comprises a coding sequence for the fusion, which contains a coding sequence for the truncated HBV core antigen at least 90% identical to SEQ ID NO: 1 or SEQ ID NO: 3, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 98%, 99% or 100% identical to SEQ ID NO: 1 or SEQ ID NO: 3, more preferably SEQ ID NO: 1 or SEQ ID NO: 3, operably linked to a coding sequence for the HBV Pol antigen at least 90% identical to SEQ ID NO: 5 or SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or SEQ ID NO: 6, preferably 98%, 99% or 100% identical to SEQ ID NO: 5 or SEQ ID NO: 6, more preferably SEQ ID NO: 5 or SEQ ID NO: 6. Preferably, the coding sequence for the truncated HBV core antigen is operably linked to the coding sequence for the HBV Pol antigen via a coding sequence for a linker at least 90% identical to SEQ ID NO: 11, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO:

11, preferably 98%, 99% or 100% identical to SEQ ID NO: 11. In particular embodiments of the application, an arenavirus vector comprises a coding sequence for the fusion having SEQ ID NO: 1 or SEQ ID NO: 3 operably linked to SEQ ID NO: 11, which is further operably linked to SEQ ID NO: 5 or SEQ ID NO: 6.

Provided herein is an expression plasmid that encodes one or more components required for the generation of a viral vector described herein. Specifically, provided herein is an expression vector that encodes an LCMV S segment wherein the ORF for the GP protein has been deleted from the S segment and has been replaced with the ORF of human HBV core or polymerase protein.

Provided herein is an expression plasmid that encodes one or more components required for the generation of a viral vector described herein. Specifically, provided herein is an expression vector that encodes an LCMV S segment wherein the ORF for the GP protein has been deleted from the S segment and has been replaced with the ORF of human HBV core or polymerase protein.

Such vector can optionally further comprise an antibiotic resistance expression cassette including a polynucleotide encoding an antibiotic resistance gene, preferably a Kan^{r} gene, more preferably a codon optimized Kan^{r} gene of at least 90% identical to SEQ ID NO: 23, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 23, preferably 100% identical to SEQ ID NO: 23, operably linked to an Ampr (bla) promoter of SEQ ID NO: 24, upstream of and operably linked to the polynucleotide encoding the antibiotic resistance gene.

Provided herein are kits comprising one or two of the vector plasmids described herein. In certain embodiments, provided herein is a kit that comprises a) an expression plasmid that comprises the nucleotide sequence of the S segment of an LCMV vector; b) an expression plasmid that comprises the nucleotide sequence of the L segment of an LCMV vector; and c) an expression plasmid that encodes the complementing functionality. In a specific embodiment, provided herein is a kit comprising a) an expression vector that comprises the nucleotide sequence of an LCMV S segment wherein the ORF for the GP protein has been deleted from the S segment and has been replaced with the ORF of human HBV core protein (e.g., having an amino acid sequence encoded by the nucleotide sequence of SEQ ID NO: 1 or 3 or an amino acid sequence that is 80%, 81%, 82%, 83%, 84%, 85%, 86%, 87%, 88%, 89%, 90%, 91%, 92%, 93%, 94%, 95%, 96%, 97%, 98%, 99%, or 100% identical to an amino acid sequence encoded by the nucleotide sequence of SEQ ID NO: 2 or 4); b) an expression plasmid that comprises the nucleotide sequence of the L segment of an LCMV vector; and c) an expression plasmid that encodes the LCMV GP protein (or a cell line that expresses LCMV GP protein).

Provided herein are kits comprising one or two of the vector plasmids described herein. In certain embodiments, provided herein is a kit that comprises a) an expression plasmid that comprises the nucleotide sequence of the S segment of an LCMV vector; b) an expression plasmid that comprises the nucleotide sequence of the L segment of an LCMV vector; and c) an expression plasmid that encodes the complementing functionality. In a specific embodiment, provided herein is a kit comprising a) an expression vector that comprises the nucleotide sequence of an LCMV S segment wherein the ORF for the GP protein has been deleted from the S segment and has been replaced with the ORF of human HBV polymerase protein (e.g., having an amino acid sequence encoded by the nucleotide sequence of SEQ ID NO: 5 or 6 or an amino acid sequence that is 80%, 81%, 82%, 83%, 84%, 85%, 86%, 87%, 88%, 89%, 90%, 91%, 92%, 93%, 94%, 95%, 96%, 97%, 98%, 99%, or 100% identical to an amino acid sequence encoded by the nucleotide sequence of SEQ ID NO: 7); b) an expression plasmid that comprises the nucleotide sequence of the L segment of an LCMV vector; and c) an expression plasmid that encodes the LCMV GP protein (or a cell line that expresses LCMV GP protein).

The polynucleotides and arenaviral vectors encoding the HBV antigens of the application can be made by the methods and processes described in the patent application number US20180319845A1, which is incorporated herein by reference in its entirety.

Infectious arenavirus vectors expressing an HBV antigen, or a combination of HBV antigens as described herein, can be used to immunize (in a preventive manner) or treat (in an immunotherapeutic manner) subjects against HBV infection.

In certain embodiments, for use with the compositions and methods provided herein is a replication-competent, trisegmented arenavirus vector. In certain embodiments, the arenavirus vector is a tri-segmented arenavirus particle comprising one L segment and two S segments or two L segments and one S segment that do not recombine into a replication-competent bi-segmented arenavirus par-ticle.

For use with the compositions and methods provided herein are tri-segmented arenavirus particles with rearrangements of their ORFs. In one aspect, for use with the compositions and methods provided herein is a tri-segmented arenavirus particle comprising one L segment and two S segments or two L segments and one S segment. In certain embodiments, the tri-segmented arenavirus particle does not recombine into a replication competent bi-seg-mented arenavirus particle. In specific embodiments, the tri-segmented arenavirus particle comprises an ORF in a position other than the wild-type position of the ORF. In yet another specific embodiment, the tri-segmented arenavirus particle comprises all four arenavirus ORFs. Thus, in certain embodiments, the tri-segmented arenavirus particle is replication competent and infectious.

Generally, arenavirus particles can be recombinantly produced by standard reverse genetic techniques as described for LCMV (L. Platz, A. Bergthaler, J. C. de la Torre, and D. D. Pinschewer, Proc Natl Acad Sci USA 103:4663-4668, 2006; A. B. Sanchez and J. C. de la Torre, Virology 350:370, 2006; E. Ortiz-Riano, B. Y. Cheng, J. C. de la Torre, L. Martinez-Sobrido. J Gen Viral. 94: 1175-88, 2013).

To generate infectious, replication-deficient arena-viruses for use with the present invention the genome of the virus can be modified. These modifications can be: i) one or more, e.g., two, three or four, of the four arenavirus ORFs (glycoprotein (GP); nucleoprotein (NP); the matrix protein Z; the RNA-dependent RNA polymerase L) are removed or functionally inactivated to prevent formation of infectious particles in normal cells albeit still allowing gene expression in arenavirus vector-infected host cells; and ii) nucleic acids coding for HBV antigens can be introduced. Infectious, replication-deficient viruses as described herein can be produced as described in International Patent Application Publication No. WO 2009/083210 (application number PCT/EP2008/010994) and International Patent Application Publication No. WO 2014/140301 (application number PCT /EP2014/055144), each of which is incorporated by reference herein in its entirety.

In certain embodiments, an arenavirus vector of the application comprises all the necessary requirements, features and sequences necessary for using such molecules as RNA vaccines, as described in US2018/0319845 and WO2017076988, the relevant content of each of which is hereby incorporated by reference in its entirety, each of which is incorporated herein by reference in its entirety.

The polynucleotides and expression vectors encoding the HBV antigens of the application can be made by any method known in the art in view of the present disclosure.

A polynucleotide can be in the form of recombinant genomic RNAs of arenavirus particles obtained by genetic and molecular techniques described in the US patents and patent applications U.S. Pat. No. 8,592,205, US20180319845, and US20180344830, which herein are incorporated in their entirety by reference.

Once generated from cDNA, the infectious, replication-deficient arenaviruses provided herein can be propagated in complementing cells. Complementing cells are cells that provide the functionality that has been eliminated from the replication-deficient arenavirus by modification of its genome (e.g., if the ORF encoding the GP protein is deleted or functionally inactivated, a complementing cell does provide the GP protein).

Owing to the removal or functional inactivation of one or more of the viral genes in arenavirus vectors (here deletion of the glycoprotein, GP, will be taken as an example), arenavirus vectors can be generated and expanded in cells providing in trans the deleted viral gene(s), e.g., the GP in the present example. Such a complementing cell line, henceforth referred to as C-cells, is generated by transfecting a mammalian cell line such as BHK-21, HEK 293, VERO or other (here BHK-21 will be taken as an example) with one or more plasmid(s) for expression of the viral gene(s) of interest (complementation plasmid, referred to as C-plasmid). The C-plasmid(s) express the viral gene(s) deleted in the arenavirus vector to be generated under control of one or more expression cassettes suitable for expression in mammalian cells, e.g., a mammalian polymerase II promoter such as the CMV or EFlalpha promoter with a polyade-nylation signal. In addition, the complementation plasmid features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Cells that can be used, e.g., BHK-21, HEK 293, MC57G or other, are kept in culture and are transfected with the complementation plasmid(s) using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing C-cell clones are identified using Western blot or flow cytometry procedures with antibodies directed against the viral protein (s) of interest. As an alternative to the use of stably transfected C-cells transient transfection of normal cells can complement the missing viral gene(s) in each of the steps where C-cells will be used below. In addition, a helper virus can be used to provide the missing functionality in trans.

For recovering of the arenavirus vector, the following procedures can be used. First day: C-cells, typically 80% confluent in M6-well plates, are transfected with a mixture of the two TF-plasmids plus the two GS-plasmids. In certain embodiments, the TF and GS plasmids can be the same, i.e. the genome sequence and transacting factors can be transcribed by T7, polI and polII promoters from one plasmid. For this one can exploit any of the commonly used strategies such as calcium-phosphate, liposome-based protocols or electroporation. 3-5 days later: The culture supernatant (arenavirus vector preparation) is harvested, aliquoted and stored at 4° C., −20° C. or −80° C. depending on how long the arenavirus vector should be stored prior to use. Then the arenavirus vector preparation's infectious titer is assessed by an immunofocus assay on C-cells.

### Compositions, Therapeutic Combinations, and Vaccines

The application also relates to compositions, therapeutic combinations, more particularly kits, and vaccines comprising one or more HBV antigens, polynucleotides, and/or vectors encoding one or more HBV antigens according to the application. Any of the HBV antigens, polynucleotides, and/or vectors of the application described herein can be used in the compositions, therapeutic combinations or kits, and vaccines of the application.

In an embodiment of the application, a composition comprises an arenavirus vector comprising a polynucleotide encoding a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4.

In an embodiment of the application, a composition comprises an arenavirus vector, comprising a polynucleotide encoding an HBV Pol antigen comprising an amino acid sequence that is at least 90% identical to SEQ ID NO: 7, preferably 100% identical to SEQ ID NO: 7.

In an embodiment of the application, a composition comprises an arenavirus vector, comprising a polynucleotide encoding a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4; and an arenavirus vector, comprising a polynucleotide encoding an HBV Pol antigen comprising an amino acid sequence that is at least 90% identical to SEQ ID NO: 7, preferably 100% identical to SEQ ID NO: 7. The arenavirus vector comprising the coding sequence for the truncated HBV core antigen and the arenavirus vector comprising the coding sequence for the HBV Pol antigen can be the same arenavirus vector, or two different arenavirus vectors.

In an embodiment of the application, a composition comprises an arenavirus vector, comprising a polynucleotide encoding a fusion protein comprising a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4, operably linked to an HBV Pol antigen comprising an amino acid sequence that is at least 90% identical to SEQ ID NO: 7, preferably 100% identical to SEQ ID NO: 7, or vice versa. Preferably, the fusion protein further comprises a linker that operably links the truncated HBV core antigen to the HBV Pol antigen, or vice versa. Preferably, the linker has the amino acid sequence of (AlaGly)n, wherein n is an integer of 2 to 5.

The application also relates to a therapeutic combination or a kit comprising an arenavirus vector expressing a truncated HBV core antigen and an HBV pol antigen according to embodiments of the application. Any arenavirus vectors encoding HBV core and pol antigens of the application described herein can be used in the therapeutic combinations or kits of the application.

In a particular embodiment of the application, a therapeutic combination or kit comprises an arenavirus vector replicon comprising: i) a first polynucleotide sequence encoding a truncated HBV core antigen consisting of an amino acid sequence that is at least 95% identical to SEQ ID NO: 2 or SEQ ID NO: 4; and ii) a second polynucleotide sequence encoding an HBV polymerase antigen having an amino acid sequence that is at least 90% identical to SEQ ID NO: 7, wherein the HBV polymerase antigen does not have reverse transcriptase activity and RNase H activity.

According to embodiments of the application, the polynucleotides in a vaccine composition or kit can be linked or separate, such that the HBV antigens expressed from such polynucleotides are fused together or produced as separate proteins, whether expressed from the same or different polynucleotides. In an embodiment, the first and second polynucleotides are present in separate vectors used in combination either in the same or separate compositions, such that the expressed proteins are also separate proteins, but used in combination. In another embodiment, the HBV antigens encoded by the first and second polynucleotides can be expressed from the same vector, e.g., such that an HBV core-pol fusion antigen is produced. Optionally, the core and pol antigens can be joined or fused together by a short linker. Alternatively, the HBV antigens encoded by the first and second polynucleotides can be expressed independently from a single vector using a ribosomal slippage site (also known as cis-hydrolase site) between the core and pol antigen coding sequences. This strategy results in a bicistronic expression vector in which individual core and pol antigens are produced from a single mRNA transcript. The core and pol antigens produced from such a bicistronic expression vector can have additional N or C-terminal residues, depending upon the ordering of the coding sequences on the mRNA transcript. Examples of ribosomal slippage sites that can be used for this purpose include, but are not limited to, the FA2 slippage site from foot-and-mouth disease virus (FMDV). Another possibility is that the HBV antigens encoded by the first and second polynucleotides can be expressed independently from two separate vectors, one encoding the HBV core antigen and one encoding the HBV pol antigen.

In a preferred embodiment, the first and second polynucleotides are present in separate arenavirus vectors. Preferably, the separate arenavirus vectors are present in the same composition.

According to preferred embodiments of the application, a therapeutic combination or kit comprises a first polynucleotide present in a first arenavirus vector, a second polynucleotide present in a second arenavirus vector. The first and second arenavirus vectors can be the same or different.

In another preferred embodiment, the first and second polynucleotides are present in a single arenavirus vector.

When a therapeutic combination of the application comprises a first arenavirus vector, and a second arenavirus vector, the amount of each of the first and second arenavirus vector is not particularly limited. For example, the first arenavirus vector and the second arenavirus vector can be present in a ratio of 10:1 to 1:10, by weight, such as 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, by weight. Preferably, the first and second arenavirus vector are present in a ratio of 1:1, by weight. The therapeutic combination of the application can further comprise a third vector encoding a third active agent useful for treating an HBV infection.

Compositions and therapeutic combinations of the application can comprise additional polynucleotides or vectors encoding additional HBV antigens and/or additional HBV antigens or immunogenic fragments thereof, such as an HBsAg, an HBV L protein or HBV envelope protein, or a polynucleotide sequence encoding thereof. However, in particular embodiments, the compositions and therapeutic combinations of the application do not comprise certain antigens.

In a particular embodiment, a composition or therapeutic combination or kit of the application does not comprise a HBsAg or a polynucleotide sequence encoding the HBsAg.

In another particular embodiment, a composition or therapeutic combination or kit of the application does not comprise an HBV L protein or a polynucleotide sequence encoding the HBV L protein.

In yet another particular embodiment of the application, a composition or therapeutic combination of the application does not comprise an HBV envelope protein or a polynucleotide sequence encoding the HBV envelope protein.

Compositions and therapeutic combinations of the application can also comprise a pharmaceutically acceptable carrier. A pharmaceutically acceptable carrier is non-toxic and should not interfere with the efficacy of the active ingredient. Pharmaceutically acceptable carriers can include one or more excipients such as binders, disintegrants, swelling agents, suspending agents, emulsifying agents, wetting agents, lubricants, flavorants, sweeteners, preservatives, dyes, solubilizers and coatings. Pharmaceutically acceptable carriers can include vehicles, such as lipid nanoparticles (LNPs). The precise nature of the carrier or other material can depend on the route of administration, e.g., intramuscular, intradermal, subcutaneous, oral, intravenous, cutaneous, intramucosal (e.g., gut), intranasal or intraperitoneal routes. For liquid injectable preparations, for example, suspensions and solutions, suitable carriers and additives include water, glycols, oils, alcohols, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For nasal sprays/inhalant mixtures, the aqueous solution/suspension can comprise water, glycols, oils, emollients, stabilizers, wetting agents, preservatives, aromatics, flavors, and the like as suitable carriers and additives.

Compositions and therapeutic combinations of the application can be formulated in any matter suitable for administration to a subject to facilitate administration and improve efficacy, including, but not limited to, oral (enteral) administration and parenteral injections. The parenteral injections include intravenous injection or infusion, subcutaneous injection, intradermal injection, and intramuscular injection. Compositions of the application can also be formulated for other routes of administration including transmucosal, ocular, rectal, long acting implantation, sublingual administration, under the tongue, from oral mucosa bypassing the portal circulation, inhalation, or intranasal.

Formulation of RNA as a conventional pharmaceutical preparation can be done using standard pharmaceutical formulation chemistries and methodologies, which are available to those skilled in the art. Any pharmaceutically acceptable carrier or excipient may be used. Auxiliary substances, such as wetting or emulsifying agents, pH buffering substances and the like, may be present in the excipient or vehicle. These excipients, vehicles and auxiliary substances are generally pharmaceutical agents which may be administered without undue toxicity and which, in the case of vaccine compositions will not induce an immune response in the individual receiving the composition. A suitable carrier can be a liposome.

Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. It is also preferred, although not required, that the preparation will contain a pharmaceutically acceptable excipient that serves as a stabilizer, particularly for peptide, protein or other like molecules if they are to be included in the composition. Examples of suitable carriers that also act as stabilizers for peptides include, without limitation, pharmaceutical grades of dextrose, sucrose, lactose, trehalose, mannitol, sorbitol, inositol, dextran, and the like.

Other suitable carriers include, again without limitation, starch, cellulose, sodium or calcium phosphates, citric acid, tartaric acid, glycine, high molecular weight polyethylene glycols (PEGs), and combination thereof. A thorough discussion of pharma-ceutically acceptable excipients, vehicles and auxiliary substances is available in REMINGTON'S PHARMACEUTICAL SCIENCES (Mack Pub. Co., N.J. 1991), incorporated herein by reference.

In a preferred embodiment of the application, compositions and therapeutic combinations of the application are formulated for parental injection, preferably subcutaneous, intradermal injection, or intramuscular injection, more preferably intramuscular injection.

According to embodiments of the application, compositions and therapeutic combinations for administration will typically comprise a buffered solution in a pharmaceutically acceptable carrier, e.g., an aqueous carrier such as buffered saline and the like, e.g., phosphate buffered saline (PBS). The compositions and therapeutic combinations can also contain pharmaceutically acceptable substances as required to approximate physiological conditions such as pH adjusting and buffering agents. For example, a composition or therapeutic combination of the application comprising an arenavirus vector can contain phosphate buffered saline (PBS) as the pharmaceutically acceptable carrier.

Compositions and therapeutic combinations of the application can be formulated as a vaccine (also referred to as an “immunogenic composition”) according to methods well known in the art. Such compositions can include adjuvants to enhance immune responses. The optimal ratios of each component in the formulation can be determined by techniques well known to those skilled in the art in view of the present disclosure.

In certain embodiments, a further adjuvant can be included in a composition or therapeutic combination of the application, or co-administered with a composition or therapeutic combination of the application. Use of another adjuvant is optional, and can further enhance immune responses when the composition is used for vaccination purposes. Other adjuvants suitable for co-administration or inclusion in compositions in accordance with the application should preferably be ones that are potentially safe, well tolerated and effective in humans. An adjuvant can be a small molecule or antibody including, but not limited to, immune checkpoint inhibitors (e.g., anti-PD1, anti-TIM-3, etc.), toll-like receptor agonists (e.g., TLR7 agonists and/or TLR8 agonists), RIG-1 agonists, IL-15 superagonists (Altor Bioscience), mutant IRF3 and IRF7 genetic adjuvants, STING agonists (Aduro), FLT3L genetic adjuvant, and IL-7-hyFc. For example, adjuvants can e.g., be chosen from among the following anti-HBV agents: HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, CTLA-4 inhibitors; Agonists of co-stimulatory receptors that are expressed on immune cells (more particularly T cells), such as CD27 and CD28; BTK inhibitors; Other drugs for treating HBV; IDO inhibitors; Arginase inhibitors; and KDM5 inhibitors.

The application also provides methods of making compositions and therapeutic combinations of the application. A method of producing a composition or therapeutic combination comprises mixing an isolated polynucleotide encoding an HBV antigen, vector, and/or polypeptide of the application with one or more pharmaceutically acceptable carriers. One of ordinary skill in the art will be familiar with conventional techniques used to prepare such compositions.

The compositions comprise the infectious arenavi-ruses described herein alone or together with a pharmaceutically acceptable carrier. Suspensions or dispersions of genetically engineered arenaviruses, especially isotonic aqueous suspensions or dispersions, can be used. The pharmaceutical compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. In certain embodiments, such dispersions or suspensions may comprise viscosity-regulating agents. The suspensions or dispersions are kept at temperatures around 2-8° C., or preferentially for longer storage may be frozen and then thawed shortly before use. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

The pharmaceutical compositions comprise from about 10³ to about 10¹¹ focus forming units of the genetically engineered arenaviruses. Unit dose forms for parenteral administration are, for example, ampoules or vials, e.g., vials containing from about 10³ to 10¹⁰ focus forming units or 10⁵ to 10¹⁵ physical particles of genetically engineered arenaviruses.

In another embodiment, a vaccine or immunogenic composition provided herein is administered to a subject by, including but not limited to, oral, intradermal, intramuscular, intraperitoneal, intravenous, topical, subcutaneous, percutaneous, intranasal and inhalation routes, and via scarification (scratching through the top layers of skin, e.g., using a bifurcated needle). Specifically, subcutaneous, intramuscular or intravenous routes can be used.

For administration intranasally or by inhalation, the preparation for use according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflators may be formulated containing a powder mix of the compound and a suitable powder base such as lactose or starch.

The dosage of the active ingredient depends upon the type of vaccination and upon the subject, and their age, weight, individual condition, the individual pharmacokinetic data, and the mode of administration.

Also provided herein are processes and uses of genetically engineered arenaviruses for the manufacture of vaccines in the form of pharmaceutical preparations, which comprise genetically engineered arenaviruses as active ingredient. The pharmaceutical compositions of the present invention are prepared in a manner known per se, for example by means of conventional mixing and/or dispersing processes.

### Methods of Inducing an Immune Response or Treating an HBV Infection

The application also provides methods of inducing an immune response against hepatitis B virus (HBV) in a subject in need thereof, comprising administering to the subject an immunogenically effective amount of a composition or immunogenic composition of the application. Any of the compositions and therapeutic combinations of the application described herein can be used in the methods of the application.

As used herein, the term “infection” refers to the invasion of a host by a disease-causing agent. A disease-causing agent is considered to be “infectious” when it is capable of invading a host and replicating or propagating within the host. Examples of infectious agents include viruses, e.g., HBV and certain species of adenovirus, prions, bacteria, fungi, protozoa and the like. “HBV infection” specifically refers to invasion of a host organism, such as cells and tissues of the host organism, by HBV.

The phrase “inducing an immune response” when used with reference to the methods described herein encompasses causing a desired immune response or effect in a subject in need thereof against an infection, e.g., an HBV infection. “Inducing an immune response” also encompasses providing a therapeutic immunity for treating against a pathogenic agent, e.g., HBV. As used herein, the term “therapeutic immunity” or “therapeutic immune response” means that the vaccinated subject is able to control an infection with the pathogenic agent against which the vaccination was done, for instance immunity against HBV infection conferred by vaccination with HBV vaccine. In an embodiment, “inducing an immune response” means producing an immunity in a subject in need thereof, e.g., to provide a therapeutic effect against a disease, such as HBV infection. In certain embodiments, “inducing an immune response” refers to causing or improving cellular immunity, e.g., T cell response, against HBV infection. In certain embodiments, “inducing an immune response” refers to causing or improving a humoral immune response against HBV infection. In certain embodiments, “inducing an immune response” refers to causing or improving a cellular and a humoral immune response against HBV infection.

As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all. Usually, a subject having a “protective immune response” or “protective immunity” against a certain agent will not die as a result of the infection with said agent.

Typically, the administration of compositions and therapeutic combinations of the application will have a therapeutic aim to generate an immune response against HBV after HBV infection or development of symptoms characteristic of HBV infection, e.g., for therapeutic vaccination.

As used herein, “an immunogenically effective amount” or “immunologically effective amount” means an amount of a composition, polynucleotide, vector, or antigen sufficient to induce a desired immune effect or immune response in a subject in need thereof. An immunogenically effective amount can be an amount sufficient to induce an immune response in a subject in need thereof. An immunogenically effective amount can be an amount sufficient to produce immunity in a subject in need thereof, e.g., provide a therapeutic effect against a disease such as HBV infection. An immunogenically effective amount can vary depending upon a variety of factors, such as the physical condition of the subject, age, weight, health, etc.; the particular application, e.g., providing protective immunity or therapeutic immunity; and the particular disease, e.g., viral infection, for which immunity is desired. An immunogenically effective amount can readily be determined by one of ordinary skill in the art in view of the present disclosure.

In particular embodiments of the application, an immunogenically effective amount refers to the amount of a composition or therapeutic combination which is sufficient to achieve one, two, three, four, or more of the following effects: (i) reduce or ameliorate the severity of an HBV infection or a symptom associated therewith; (ii) reduce the duration of an HBV infection or symptom associated therewith; (iii) prevent the progression of an HBV infection or symptom associated therewith; (iv) cause regression of an HBV infection or symptom associated therewith; (v) prevent the development or onset of an HBV infection, or symptom associated therewith; (vi) prevent the recurrence of an HBV infection or symptom associated therewith; (vii) reduce hospitalization of a subject having an HBV infection; (viii) reduce hospitalization length of a subject having an HBV infection; (ix) increase the survival of a subject with an HBV infection; (x) eliminate an HBV infection in a subject; (xi) inhibit or reduce HBV replication in a subject; and/or (xii) enhance or improve the prophylactic or therapeutic effect(s) of another therapy.

An immunogenically effective amount can also be an amount sufficient to reduce HBsAg levels consistent with evolution to clinical seroconversion; achieve sustained HBsAg clearance associated with reduction of infected hepatocytes by a subject's immune system; induce HBV-antigen specific activated T-cell populations; and/or achieve persistent loss of HBsAg within 12 months. Examples of a target index include lower HBsAg below a threshold of 500 copies of HBsAg international units (IU) and/or higher CD8 counts.

It is expected that the amount will fall in a relatively broad range that can be determined through routine trials.

An immunogenically effective amount can be from one vector, or from multiple vectors. An immunogenically effective amount can be administered in a single composition, or in multiple compositions, such as 1, 2, 3, 4, 5, 6, 7, 8, 9, or 10 compositions (e.g., tablets, capsules or injectables, or any composition adapted to intradermal delivery, e.g., to intradermal delivery using an intradermal delivery patch), wherein the administration of the multiple capsules or injections collectively provides a subject with an immunogenically effective amount. It is also possible to administer an immunogenically effective amount to a subject, and subsequently administer another dose of an immunogenically effective amount to the same subject, in a so-called prime-boost regimen. This general concept of a prime-boost regimen is well known to the skilled person in the vaccine field. Further booster administrations can optionally be added to the regimen, as needed.

A therapeutic combination comprising two arenavirus vectors, e.g., a first arenavirus vector encoding an HBV core antigen and second arenavirus vector encoding an HBV pol antigen, can be administered to a subject by mixing both replicons and delivering the mixture to a single anatomic site. Alternatively, two separate immunizations each delivering a single expression replicon can be performed. In such embodiments, whether both replicons are administered in a single immunization as a mixture of in two separate immunizations, the first arenavirus vector and the second arenavirus vector can be administered in a ratio of 10:1 to 1:10, by weight, such as 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, by weight. Preferably, the first and second arenavirus vectors are administered in a ratio of 1:1, by weight.

Preferably, a subject to be treated according to the methods of the application is an HBV-infected subject, particular a subject having chronic HBV infection. Acute HBV infection is characterized by an efficient activation of the innate immune system complemented with a subsequent broad adaptive response (e.g., HBV-specific T-cells, neutralizing antibodies), which usually results in successful suppression of replication or removal of infected hepatocytes. In contrast, such responses are impaired or diminished due to high viral and antigen load, e.g., HBV envelope proteins are produced in abundance and can be released in sub-viral particles in 1,000-fold excess to infectious virus.

Chronic HBV infection is described in phases characterized by viral load, liver enzyme levels (necroinflammatory activity), HBeAg, or HBsAg load or presence of antibodies to these antigens. cccDNA levels stay relatively constant at approximately 10 to 50 copies per cell, even though viremia can vary considerably. The persistence of the cccDNA species leads to chronicity. More specifically, the phases of chronic HBV infection include: (i) the immune-tolerant phase characterized by high viral load and normal or minimally elevated liver enzymes; (ii) the immune activation HBeAg-positive phase in which lower or declining levels of viral replication with significantly elevated liver enzymes are observed; (iii) the inactive HBsAg carrier phase, which is a low replicative state with low viral loads and normal liver enzyme levels in the serum that can follow HBeAg seroconversion; and (iv) the HBeAg-negative phase in which viral replication occurs periodically (reactivation) with concomitant fluctuations in liver enzyme levels, mutations in the pre-core and/or basal core promoter are common, such that HBeAg is not produced by the infected cell.

As used herein, “chronic HBV infection” refers to a subject having the detectable presence of HBV for more than 6 months. A subject having a chronic HBV infection can be in any phase of chronic HBV infection. Chronic HBV infection is understood in accordance with its ordinary meaning in the field. Chronic HBV infection can for example be characterized by the persistence of HBsAg for 6 months or more after acute HBV infection. For example, a chronic HBV infection referred to herein follows the definition published by the Centers for Disease Control and Prevention (CDC), according to which a chronic HBV infection can be characterized by laboratory criteria such as: (i) negative for IgM antibodies to hepatitis B core antigen (IgM anti-HBc) and positive for hepatitis B surface antigen (HBsAg), hepatitis B e antigen (HBeAg), or nucleic acid test for hepatitis B virus DNA, or (ii) positive for HBsAg or nucleic acid test for HBV DNA, or positive for HBeAg two times at least 6 months apart.

Preferably, an immunogenically effective amount refers to the amount of a composition or therapeutic combination of the application which is sufficient to treat chronic HBV infection.

In some embodiments, a subject having chronic HBV infection is undergoing nucleoside analog (NUC) treatment, and is NUC-suppressed. As used herein, “NUC-suppressed” refers to a subject having an undetectable viral level of HBV and stable alanine aminotransferase (ALT) levels for at least six months. Examples of nucleoside/nucleotide analog treatment include HBV polymerase inhibitors, such as entacavir and tenofovir. Preferably, a subject having chronic HBV infection does not have advanced hepatic fibrosis or cirrhosis. Such subject would typically have a METAVIR score of less than 3 for fibrosis and a fibroscan result of less than 9 kPa. The METAVIR score is a scoring system that is commonly used to assess the extent of inflammation and fibrosis by histopathological evaluation in a liver biopsy of patients with hepatitis B. The scoring system assigns two standardized numbers: one reflecting the degree of inflammation and one reflecting the degree of fibrosis.

It is believed that elimination or reduction of chronic HBV can allow early disease interception of severe liver disease, including virus-induced cirrhosis and hepatocellular carcinoma. Thus, the methods of the application can also be used as therapy to treat HBV-induced diseases. Examples of HBV-induced diseases include, but are not limited to cirrhosis, cancer (e.g., hepatocellular carcinoma), and fibrosis, particularly advanced fibrosis characterized by a METAVIR score of 3 or higher for fibrosis. In such embodiments, an immunogenically effective amount is an amount sufficient to achieve persistent loss of HBsAg within 12 months and significant decrease in clinical disease (e.g., cirrhosis, hepatocellular carcinoma, etc.).

Methods according to embodiments of the application further comprises administering to the subject in need thereof another immunogenic agent (such as another HBV antigen or other antigen) or another anti-HBV agent (such as a nucleoside analog or other anti-HBV agent) in combination with a composition of the application. For example, another anti-HBV agent or immunogenic agent can be a small molecule or antibody including, but not limited to, immune checkpoint inhibitors (e.g., anti-PD1, anti-TIM-3, etc.), toll-like receptor agonists (e.g., TLR7 agonists and/oror TLR8 agonists), RIG-1 agonists, IL-15 superagonists (Altor Bioscience), mutant IRF3 and IRF7 genetic adjuvants, STING agonists (Aduro), FLT3L genetic adjuvant, IL12 genetic adjuvant, IL-7-hyFc; CAR-T which bind HBV env (S-CAR cells); capsid assembly modulators; cccDNA inhibitors, HBV polymerase inhibitors (e.g., entecavir and tenofovir). The one or other anti-HBV active agents can be, for example, a small molecule, an antibody or antigen binding fragment thereof, a polypeptide, protein, or nucleic acid. The one or other anti-HBV agents can e.g., be chosen from among HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, and CTLA-4 inhibitors; Agonists of co-stimulatory receptors that are expressed on immune cells (more particularly T cells), such as CD27, CD28; BTK inhibitors; Other drugs for treating HBV; IDO inhibitors; Arginase inhibitors; and KDMS inhibitors.

In yet another embodiment, provided herein is the combined use of the replication-deficient arenavirus expressing an HBV antigen described herein and one or more replication-defective virus vectors. In a more specific embodiment the replication-defective virus vector is selected from the group comprising of poxviruses, adeno-viruses, alphaviruses, herpes simplex viruses, paramyxoviruses, rhabdoviruses, poliovirus, adeno-associated virus, and sendai virus, and mixtures thereof. In a specific embodiment, the poxvirus is a modified vaccine Ankara.

In yet another embodiment, provided herein is the combined use of the replication-deficient arenavirus expressing an HBV antigen described herein and one or more replication-defective virus vectors expressing an HBV antigen. In a more specific embodiment the replication-defective virus vector is selected from the group comprising of poxviruses, adenoviruses, alphaviruses, herpes simplex viruses, paramyxoviruses, rhabdoviruses, poliovirus, adeno-associated virus, and sendai virus, and mixtures thereof. In a specific embodiment, the poxvirus is a modified vaccine Ankara.

In another embodiment, the first infectious arenavirus expressing an HBV antigen as described herein is administered before or after the second infectious arenavirus expressing an HBV antigen as described herein. For example the first infectious arenavirus expressing an HBV antigen is administered around 30-60 minutes before or after the first administration of the second infectious arenavirus.

In another embodiment, the first infectious arenavirus expressing a vaccine antigen is administered before the second infectious arenavirus expressing a vaccine antigen. In certain embodiments there is a period of about 1 hour, 2 hours, 3 hours, 6 hours, 12 hours, 1 day, 2 days, 3 days, 5 days, 1 week, 2 weeks, 1 month, 2 months, 3 months, 4 months, 5 months, 6 months, 7 months, 8 months, 9 months, 10 months, 11 months, 1 year between the administration of the first infectious arenavirus and the second infectious arenavirus.

In another embodiment, two infectious arenaviruses are administered in a treatment regime at molar ratios ranging from about 1: 1 to 1: 1000, in particular including: 1: 1 ratio, 1:2 ratio, 1:5 ratio, 1: 10 ratio, 1:20 ratio, 1:50 ratio, 1:100 ratio, 1:200 ratio, 1:300 ratio, 1:400 ratio, 1:500 ratio, 1:600 ratio, 1:700 ratio, 1:800 ratio, 1:900 ratio, 1:1000 ratio.

In another embodiment, administering two or more infectious arenaviruses expressing an HBV antigen, administered sequentially, reduces the risk that an individual will develop an infection with HBV by at least 10%, at least about 20%, at least about 25%, at least about 30%, at least about 40%, at least about 50%, at least about 60%, at least about 70%, at least about 80%, at least about 90%, or more, compared to the risk of developing an infection with HBV in the absence of such treatment.

In one aspect, provided herein are such genetically modified replication-deficient arenaviruses suitable as vaccines and methods of using such arenaviruses in vaccination and treatment or prevention of infections by HBV.

In certain embodiments, immunization with an infectious arenavirus that expresses an HBV antigen or a fragment thereof, as described herein provides a long-lasting immune response. In certain embodiments, maximal antibody levels can be achieved after two immunizations. In another embodiment, a third immunization can be administered for a boosting effect. In more specific embodiments, provided herein are administration schedules using the infectious arenavirus in a vaccination for the treatment and/or prevention of infections by HBV. In certain embodiments, the infectious arenavirus viral vector is replication-deficient. In certain embodiments, the infectious arenavirus viral vector is replication-competent.

In certain embodiments, administering to a seronegative subject an infectious arenavirus expressing an HBV antigen or a fragment thereof, as described herein induces a detectable antibody titer for a minimum of at least 4 weeks. In another embodiment, administering to a subject infected with an HBV infection an infectious arenavirus expressing an HBV antigen or a fragment thereof, as described herein increases the antibody titer by at least 100%, at least 200%, at least 300%, at least 400%, at least 500%, or at least 1000%. In certain embodiments, primary antigen exposure, by first immunization with an infectious arenavirus expressing an HBV antigen, elicits a functional, (neutralizing) and minimum antibody titer of at least 50%, at least 100%, at least 200%, at least 300%, at least 400%, at least 500%, or at least 1000% of mean control sera from infection-immune human subjects. In more specific embodiments, the primary neutralizing geometric mean antibody titer increases up to a peak value of at least 1:50, at least 1:100, at least 1:200, or at least 1:1000 within at least 4 weeks post-immunization. In another embodiment, immunization with an infectious arenavirus expressing an HBV antigen or a fragment thereof, as described herein produces high titers of antibodies that last for at least 4 weeks, at least 8 weeks, at least 12 weeks, at least 6 months, at least 12 months, at least 2 years, at least 3 years, at least 4 years, or at least 5 years post-immunization following a single administration of the vaccine. In certain embodiments, the infectious arenavirus viral vector is replication-deficient. In certain embodiments, the infectious arenavirus viral vector is replication-competent.

In yet another embodiment, secondary antigen exposure by second immunization with an infectious arenavirus expressing an HBV antigen or a fragment thereof increases the antibody titer by at least 100%, at least 200%, at least 300%, at least 400%, at least 500%, or at least 1000%. In another embodiment, secondary antigen exposure elicits a functional, (neutralizing) and minimum antibody titer of at least 50%, at least 100%, at least 200%, at least 300%, at least 400%, at least 500%, or at least 1000% of mean control sera from infection-immune human subjects. In more specific embodiments, the secondary neutralizing geometric mean antibody titer increases up to a peak value of at least 1:50, at least 1:100, at least 1:200, or at least 1:1000 within at least 4 weeks post-immunization. In another embodiment, a second immunization with an infectious arenavirus expressing an HBV antigen or a fragment thereof, as described herein produces high titers of antibodies that last for at least 4 weeks, at least 8 weeks, at least 12 weeks, at least 6 months, at least 12 months, at least 2 years, at least 3 years, at least 4 years, or at least 5 years post-immunization. In certain embodiments, the infectious arenavirus viral vector is replication-deficient. In certain embodiments, the infectious arenavirus viral vector is replication-competent.

In yet another embodiment, a third boosting immunization increases the antibody titer by at least 100%, at least 200%, at least 300%, at least 400%, at least 500%, or at least 1000%. In another embodiment, the boosting immunization elicits a functional, (neutralizing) and minimum antibody titer of at least 50%, at least 100%, at least 200%, at least 300%, at least 400%, at least 500%, or at least 1000% of mean control sera from infection-immune human subjects. In more specific embodiments, the neutralizing geometric mean antibody titer after the third boosting immunization increases up to a peak value of at least 1:50, at least 1:100, at least 1:200, or at least 1:1000 within at least 4 weeks post-immunization. In another embodiment, a third boosting immunization prolongs the antibody titer by at least 4 weeks, at least 8 weeks, at least 12 weeks, at least 6 months, at least 12 months, at least 2 years, at least 3 years, at least 4 years, or at least 5 years post-immunization.

In certain embodiments, the infectious arenavirus expressing an HBV antigen or fragment thereof, elicits a T cell independent or T cell dependent response. In other embodiments, the infectious arenavirus expressing an HBV antigen or a fragment thereof, elicits a T cell response. In other embodiments, the infectious arenavirus expressing an HBV antigen or a fragment thereof, as described herein elicits a T helper response. In another embodiment, the infectious arenavirus expressing an HBV antigen or a fragment thereof, as described herein elicits a Th1-orientated response or a Th2-orientated response. In certain embodiments, the infectious arenavirus viral vector is replication-deficient. In certain embodiments, the infectious arenavirus viral vector is replication-competent.

In more specific embodiments, the Th1-orientated response is indicated by a predominance of IgG1 antibodies versus IgG2. In other embodiments the ratio of IgG1:IgG2 is greater than 1:1, greater than 2:1, greater than 3:1, or greater than 4:1. In another embodiment the infectious arenavirus expressing an HBV antigen or a fragment thereof, as described herein is indicated by a predominance of IgG3 antibodies. In certain embodiments, the infectious arenavirus viral vector is replication-deficient. In certain embodiments, the infectious arenavirus viral vector is replication-competent.

In some embodiments, the infectious arenavirus expressing an HBV antigen or a fragment thereof elicits a CD8+ T cell response. In other embodiments, the infectious arenavirus expressing an HBV antigen or a fragment thereof elicits a regulatory T cell response. In more specific embodiments, the regulatory T cell response maintains immune tolerance. In another embodiment, the infectious arenavirus expressing an HBV antigen or a fragment thereof elicits both CD4+ and CD8+ T cell responses. In certain embodiments, the infectious arenavirus viral vector is replication-deficient. In certain embodiments, the infectious arenavirus viral vector is replication-competent.

In certain embodiments, the infectious arenavirus expressing one or more HBV antigens or fragment thereof, as described herein, elicits high titers of neutralizing antibodies. In another embodiment, the infectious arenavirus expressing two or more HBV antigens or fragments thereof, as described herein, elicits higher titers of neutralizing antibodies than expression of the protein complex components individually. In certain embodiments, the infectious arenavirus viral vector is replication-deficient. In certain embodiments, the infectious arenavirus viral vector is replication-competent.

In other embodiments, two or more infectious arenaviruses expressing an HBV antigen elicit high titers of neutralizing antibodies. In a more specific embodiment, two or more infectious arenaviruses expressing an HBV antigen elicit higher titers of neutralizing antibodies than an infectious arenavirus expressing one HBV antigen or fragment thereof. In certain embodiments, the infectious arenavirus viral vector is replication-deficient. In certain embodiments, the infectious arenavirus viral vector is replication-competent.

In another embodiment, the infectious arenavirus expressing two, three, four, five, or more HBV antigens elicits higher titers of neutralizing antibodies than an infectious arenavirus expressing one HBV antigen or fragment thereof. In certain embodiments, the infectious arenavirus viral vector is replication-deficient. In certain embodiments, the infectious arenavirus viral vector is replication-competent.

### Methods of Delivery

Compositions and therapeutic combinations of the application can be administered to a subject by any method known in the art in view of the present disclosure, including, but not limited to, parenteral administration (e.g., intramuscular, subcutaneous, intravenous, or intradermal injection), oral administration, transdermal administration, and nasal administration. Preferably, compositions and therapeutic combinations are administered parenterally (e.g., by intramuscular injection or intradermal injection) or transdermally.

### Adjuvants

In some embodiments of the application, a method of inducing an immune response against HBV further comprises administering an adjuvant. The terms “adjuvant” and “immune stimulant” are used interchangeably herein and are defined as one or more substances that cause stimulation of the immune system. In this context, an adjuvant is used to enhance an immune response to HBV antigens and antigenic HBV polypeptides of the application.

According to embodiments of the application, an adjuvant can be present in a therapeutic combination or composition of the application or administered in a separate composition. An adjuvant can be, e.g., a small molecule or an antibody. Examples of adjuvants suitable for use in the application include, but are not limited to, immune checkpoint inhibitors (e.g., anti-PD1, anti-TIM-3, etc.), toll-like receptor agonists (e.g., TLR7 and/or TLR8 agonists), RIG-1 agonists, IL-15 superagonists (Altor Bioscience), mutant IRF3 and IRF7 genetic adjuvants, STING agonists (Aduro), FLT3L genetic adjuvant, IL12 genetic adjuvant, and IL-7-hyFc. Examples of adjuvants can e.g., be chosen from among the following anti-HBV agents: HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L 1 inhibitors, PD-1 inhibitors, TIM-3 inhibitors, TIGIT inhibitors, Lag3 inhibitors, and CTLA-4 inhibitors; Agonists of co-stimulatory receptors that are expressed on immune cells (more particularly T cells), such as CD27, CD28; BTK inhibitors; Other drugs for treating HBV; IDO inhibitors; Arginase inhibitors; and KDMS inhibitors.

Compositions and therapeutic combinations of the application can also be administered in combination with at least one other anti-HBV agent. Examples of anti-HBV agents suitable for use with the application include, but are not limited to small molecules, antibodies, and/or CAR-T therapies which bind HBV env (S-CAR cells), capsid assembly modulators, TLR agonists (e.g., TLR7 and/or TLR8 agonists), cccDNA inhibitors, HBV polymerase inhibitors (e.g., entecavir and tenofovir), and/or immune checkpoint inhibitors, etc.

The at least one anti-HBV agent can e.g., be chosen from among HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, and CTLA-4 inhibitors; Agonists of co-stimulatory receptors that are expressed on immune cells (more particularly T cells), such as CD27, CD28; BTK inhibitors; Other drugs for treating HBV; IDO inhibitors; Arginase inhibitors; and KDMS inhibitors. Such anti-HBV agents can be administered with the compositions and therapeutic combinations of the application simultaneously or sequentially.

### Methods of Prime/Boost Immunization

Embodiments of the application also contemplate administering an immunogenically effective amount of a composition or therapeutic combination to a subject, and subsequently administering another dose of an immunogenically effective amount of a composition or therapeutic combination to the same subject, in a so-called prime-boost regimen Thus, in an embodiment, a composition or therapeutic combination of the application is a primer vaccine used for priming an immune response. In another embodiment, a composition or therapeutic combination of the application is a booster vaccine used for boosting an immune response. The priming and boosting vaccines of the application can be used in the methods of the application described herein. This general concept of a prime-boost regimen is well known to the skilled person in the vaccine field. Any of the compositions and therapeutic combinations of the application described herein can be used as priming and/or boosting vaccines for priming and/or boosting an immune response against HBV.

In some embodiments of the application, a composition or therapeutic combination of the application can be administered for priming immunization. The composition or therapeutic combination can be re-administered for boosting immunization. Further booster administrations of the composition or vaccine combination can optionally be added to the regimen, as needed. An adjuvant can be present in a composition of the application used for boosting immunization, present in a separate composition to be administered together with the composition or therapeutic combination of the application for the boosting immunization, or administered on its own as the boosting immunization. In those embodiments in which an adjuvant is included in the regimen, the adjuvant is preferably used for boosting immunization.

An illustrative and non-limiting example of a prime-boost regimen includes administering a single dose of an immunogenically effective amount of a composition or therapeutic combination of the application to a subject to prime the immune response; and subsequently administering another dose of an immunogenically effective amount of a composition or therapeutic combination of the application to boost the immune response, wherein the boosting immunization is first administered about two to six weeks, preferably four weeks after the priming immunization is initially administered. Optionally, about 10 to 14 weeks, preferably 12 weeks, after the priming immunization is initially administered, a further boosting immunization of the composition or therapeutic combination, or other adjuvant, is administered.

In certain embodiments, provided herein are methods for treating and/or preventing an HBV infection comprising administering two or more arenavirus vector constructs each expressing the same or a different HBV antigen sequentially. The time interval between each administration can be about 1 week, about 2 weeks, about 3 week, about 4 weeks, about 5 weeks, about 6 weeks, about 7 weeks, about 8 weeks, about 3 months, about 4 months, about 5 months, about 6 months, about 7 months, about 8 months, about 9 months, about 10 months, about 11 months, about 12 months, about 18 months, or about 24 months.

In certain embodiments, the first infectious arena-virus and the second infectious arenavirus are homologous. In certain embodiments, the first infectious arenavirus and the second infectious arenavirus are heterologous.

In certain specific embodiments, the first infectious arenavirus is an Old World arenavirus, and the second infectious arenavirus is an Old World arenavirus. In certain specific embodiments, the first infectious arenavirus is an Old World arenavirus, and the second infectious arenavirus is a New World arenavirus. In certain specific embodiments, the first infectious arenavirus is a New World arenavirus, and the second infectious arenavirus is a New World arenavirus. In certain specific embodiments, the first infectious arenavirus is a New World arenavirus, and the second infectious arenavirus is an Old World arenavirus.

In certain specific embodiments, the first infectious arenavirus is derived from LCMV, and the second infectious arenavirus is derived from LCMV. In certain specific embodiments, the first infectious arenavirus is derived from LCMV, and the second infectious arenavirus is derived from Junin virus. In certain specific embodiments, the first infectious arenavirus is derived from Junin virus, and the second infectious arenavirus is derived from Junin virus. In certain specific embodiments, the first infectious arenavirus is derived from Junin virus, and the second infectious arenavirus is derived from LCMV.

In certain embodiments, provided herein is a method of treating and/or preventing an HBV infection wherein a first infectious arenavirus is administered first as a “prime,” and a second infectious arenavirus is administered as a “boost.” The first and the second infectious arenavirus vectors can express the same or different HBV antigens. In certain specific embodiments, the “prime” administration is performed with an infectious arenavirus derived from LCMV, and the “boost” is performed with an infectious arenavirus derived from Junin virus. In certain specific embodiments, the “prime” administration is performed with an infectious arenavirus derived from Junin virus, and the “boost” is performed with an infectious arenavirus derived from LCMV.

In certain embodiments, administering a first infectious arenavirus expressing an HBV antigen or a fragment thereof, followed by administering a second infectious arenavirus expressing an HBV antigen or a fragment thereof results in a greater antigen specific CD8+ T cell response than administering a single infectious arenavirus expressing an HBV antigen or a fragment thereof. In certain embodiments, the antigen specific CD8+ T cell count increases by 50%, 100%, 150% or 200% after the second administration compared to the first administration. In certain embodiments, administering a third infectious arenavirus expressing an HBV antigen results in a greater antigen specific CD8+ T cell response than administering two consecutive infectious arenaviruses expressing an HBV antigen. In certain embodiments, the antigen specific CD8+ T cell count increases by about 50%, about 100%, about 150%, about 200% or about 250% after the third administration compared to the first administration.

In certain embodiments, provided herein are methods for treating and/or preventing an infection comprising administering two or more arenavirus vector constructs, wherein the two or more arenavirus vector constructs are homologous, and wherein the time interval between each administration is about 1 week, about 2 weeks, about 3 week, about 4 weeks, about 5 weeks, about 6 weeks, about 7 weeks, about 8 weeks, about 3 months, about 4 months, about 5 months, about 6 months, about 7 months, about 8 months, about 9 months, about 10 months, about 11 months, about 12 months, about 18 months, or about 24 months.

In certain embodiments, administering a first infectious arenavirus expressing an HBV antigen or a fragment thereof and a second, heterologous, infectious arenavirus expressing an HBV antigen or a fragment thereof elicits a greater CDS+ T cell response than administering a first infectious arenavirus expressing an HBV antigen or a fragment thereof and a second, homologous, infectious arena-virus expressing an HBV antigen or a fragment thereof.

### Kits

Also provided herein is a kit comprising an arenavirus vector of the application. A kit can comprise an arenavirus vector encoding the first polynucleotide and an arenavirus vector encoding the second polynucleotide in one or more separate compositions, or a kit can comprise an arenavirus vector encoding the first polynucleotide and an arenavirus vector encoding the second polynucleotide in a single composition. A kit can further comprise one or more adjuvants or immune stimulants, and/or other anti-HBV agents.

The ability to induce or stimulate an anti-HBV immune response upon administration in an animal or human organism can be evaluated either in vitro or in vivo using a variety of assays which are standard in the art. For a general description of techniques available to evaluate the onset and activation of an immune response, see for example Coligan et al. (1992 and 1994, Current Protocols in Immunology; ed. J Wiley & Sons Inc, National Institute of Health). Measurement of cellular immunity can be performed by measurement of cytokine profiles secreted by activated effector cells including those derived from CD4+ and CD8+ T-cells (e.g. quantification of IL-10 or IFN gamma-producing cells by ELISPOT), by determination of the activation status of immune effector cells (e.g. T cell proliferation assays by a classical [3H] thymidine uptake or flow cytometry-based assays), by assaying for antigen-specific T lymphocytes in a sensitized subject (e.g. peptide-specific lysis in a cytotoxicity assay, etc.).

The ability to stimulate a cellular and/or a humoral response can be determined by antibody binding and/or competition in binding (see for example Harlow, 1989, Antibodies, Cold Spring Harbor Press). For example, titers of antibodies produced in response to administration of a composition providing an immunogen can be measured by enzyme-linked immunosorbent assay (ELISA). The immune responses can also be measured by neutralizing antibody assay, where a neutralization of a virus is defined as the loss of infectivity through reaction/inhibition/neutralization of the virus with specific antibody. The immune response can further be measured by Antibody-Dependent Cellular Phagocytosis (ADCP) Assay.

### EMBODIMENTS

The invention provides also the following non-limiting embodiments.

Embodiment 1 is an arenavirus vector, comprising at least one of:
a) a first polynucleotide sequence encoding the truncated HBV core antigen consisting of an amino acid sequence that is at least 95% identical to SEQ ID NO: 2 or SEQ ID NO: 4; and
b) a second polynucleotide sequence encoding the HBV polymerase antigen consisting of an amino acid sequence that is at least 90% identical to SEQ ID NO: 7, wherein the HBV polymerase antigen does not have reverse transcriptase activity and RNase H activity.

Embodiment 1a is the arenavirus vector of embodiment 1, wherein the arenavirus vector is infectious, and wherein an open reading frame that encodes a glycoprotein of the arenavirus is deleted or functionally inactivated.

Embodiment 2 is the arenavirus vector of any one of embodiments 1-1a, comprising the first polynucleotide sequence encoding a truncated HBV core antigen consisting of an amino acid sequence that is at least 95% identical to SEQ ID NO: 2 or SEQ ID NO: 4.

Embodiment 3 is the arenavirus vector of embodiment 2, comprising the second polynucleotide encoding the HBV polymerase antigen consisting of an amino acid sequence that is at least 90% identical to SEQ ID NO: 7, wherein the HBV polymerase antigen does not have reverse transcriptase activity and RNase H activity.

Embodiment 4 is the arenavirus vector of embodiment 3, comprising:
a) a first polynucleotide sequence encoding a truncated HBV core antigen consisting of the amino acid sequence of SEQ ID NO: 2 or SEQ ID NO: 4; and
b) a second polynucleotide sequence encoding the HBV polymerase antigen comprising the amino acid sequence of SEQ ID NO: 7, wherein the HBV polymerase antigen does not have reverse transcriptase activity and RNase H activity.

Embodiment 5 the arenavirus vector of any one of embodiments 1-4, wherein the first polynucleotide further comprises a polynucleotide sequence encoding a signal sequence operably linked to the N-terminus of the truncated HBV core antigen.

Embodiment 5a is the arenavirus vector of any one of embodiments 1-5, wherein the second polynucleotide further comprises further comprises a polynucleotide sequence encoding a signal sequence operably linked to the N-terminus of the HBV polymerase antigen.

Embodiment 5b is the arenavirus vector of embodiment 5 or 5a, wherein the signal sequence independently comprises the amino acid sequence of SEQ ID NO: 9 or SEQ ID NO: 15.

Embodiment 5c is the arenavirus vector of embodiment 5 or 5a, wherein the signal sequence is independently encoded by the polynucleotide sequence of SEQ ID NO: 8 or SEQ ID NO: 14.

Embodiment 6 is the arenavirus vector of any one of embodiments 1-5c, wherein the HBV polymerase antigen comprises an amino acid sequence that is at least 98%, such as at least 98%, 98.5%, 99%, 99.1%, 99.2%, 99.3%, 99.4%, 99.5%, 99.6%, 99.7%, 99.8%, 99.9%, or 100%, identical to SEQ ID NO: 7.

Embodiment 6a is the arenavirus vector of embodiment 6, wherein the HBV polymerase antigen comprises the amino acid sequence of SEQ ID NO: 7.

Embodiment 6b is the arenavirus vector of any one of embodiments 1 to 6a, wherein and the truncated HBV core antigen consists of the amino acid sequence that is at least 98%, such as at least 98%, 98.5%, 99%, 99.1%, 99.2%, 99.3%, 99.4%, 99.5%, 99.6%, 99.7%, 99.8%, 99.9%, or 100%, identical to SEQ ID NO: 2 or SEQ ID NO: 4.

Embodiment 6c is the arenavirus vector of embodiment 6b, wherein the truncated HBV antigen consists of the amino acid sequence of SEQ ID NO: 2 or SEQ ID NO: 4.

Embodiment 7 is the arenavirus vector of any one of embodiments 1-6c, wherein the first polynucleotide sequence comprises a polynucleotide sequence having at least 90%, such as at least 90%, 91%, 92%, 93%, 94%, 95%, 96%, 97%, 98%, 99% or 100%, sequence identity to SEQ ID NO: 1 or SEQ ID NO: 3.

Embodiment 7a is the arenavirus vector of embodiment 7, wherein the first polynucleotide sequence comprises a polynucleotide sequence having at least 98%, such as at least 98%, 98.5%, 99%, 99.1%, 99.2%, 99.3%, 99.4%, 99.5%, 99.6%, 99.7%, 99.8%, 99.9%, or 100%, sequence identity to SEQ ID NO: 1 or SEQ ID NO: 3.

Embodiment 8 is the arenavirus vector of embodiment 7a, wherein the first polynucleotide sequence comprises the polynucleotide sequence of SEQ ID NO: 1 or SEQ ID NO: 3.

Embodiment 9 the arenavirus vector of any one of embodiments 1 to 8, wherein the second polynucleotide sequence comprises a polynucleotide sequence having at least 90%, such as at least 90%, 91%, 92%, 93%, 94%, 95%, 96%, 97%, 98%, 99% or 100%, sequence identity to SEQ ID NO: 5 or SEQ ID NO: 6.

Embodiment 9a the arenavirus vector of embodiment 9, wherein the second polynucleotide sequence comprises a polynucleotide sequence having at least 98%, such as at least 98%, 98.5%, 99%, 99.1%, 99.2%, 99.3%, 99.4%, 99.5%, 99.6%, 99.7%, 99.8%, 99.9%, or 100%, sequence identity to SEQ ID NO: 5 or SEQ ID NO: 6.

Embodiment 10 is the arenavirus vector of embodiment 9a, wherein the second polynucleotide sequence comprises the polynucleotide sequence of SEQ ID NO: 5 or SEQ ID NO: 6.

Embodiment 11 is the arenavirus vector of any one of embodiments 1 to 10, encoding a fusion protein comprising the truncated HBV core antigen operably linked to the HBV polymerase antigen.

Embodiment 12 is the arenavirus vector of embodiment 11, wherein the fusion protein comprises the truncated HBV core antigen operably linked to the HBV polymerase antigen via a linker.

Embodiment 13 is the arenavirus vector of embodiment 12, wherein the linker comprises the amino acid sequence of (AlaGly)n, and n is an integer of 2 to 5.

Embodiment 13a is the arenavirus vector of embodiment 13, wherein the linker is encoded by a polynucleotide sequence at least 90% identical to SEQ ID NO: 11, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 11.

Embodiment 13b is the arenavirus vector of embodiment 13a, wherein the linker is encoded by a polynucleotide sequence comprising SEQ ID NO: 11.

Embodiment 14 is the arenavirus vector of any one of embodiments 13-13b, wherein the fusion protein comprises the amino acid sequence of SEQ ID NO: 16.

Embodiment 15 is the arenavirus vector of any one of embodiments 1-14, wherein the arenavirus vector is replication-deficient, has the ability to amplify and express its genetic information in infected cells but is unable to produce further infectious progeny particles in normal, not genetically engineered cells.

Embodiment 15a is the arenavirus vector of embodiment 15, wherein the open reading frame that encodes the glycoprotein of the arenavirus is deleted.

Embodiment 15b is the arenavirus vector of embodiment 15 or 15a, wherein the genomic information encoding the infectious arenavirus viral vector is derived from the lymphocytic choriomeningitis virus Clone 13 strain.

Embodiment 15c is the arenavirus vector of embodiment 15 or 15a, wherein the genomic information encoding the infectious arenavirus viral vector is derived from the lymphocytic choriomeningitis MP strain.

Embodiment 15d is the arenavirus vector of any one of embodiments 15 to 15c, wherein the viral vector comprises a genomic segment, wherein the genomic segment comprises a nucleotide sequence that is at least 90%, 91%, 92%, 93%, 94%, 95%, 96%, 97%, 98%, at least 99%, or 100% identical to the sequence of nucleotide 1639 to 3315 of SEQ ID NO: 29 or 1640 to 3316 of SEQ ID NO: 25.

Embodiment 15d is the arenavirus vector of any one of embodiments 15 to 15c, wherein the viral vector comprises a genomic segment comprising a nucleotide sequence encoding an expression product whose amino acid sequence is at least 90%, 91%, 92%, 93%, 94%, 95%, 96%, 97%, 98%, at least 99%) or 100% identical to the amino acid sequence encoded by 1639 to 3315 of SEQ ID NO: 29 or 1640 to 3316 of SEQ ID NO: 25.

Embodiment 16 is the arenavirus vector of embodiment 15 or 15a, wherein the arenavirus is Junin virus.

Embodiment 16a is the arenavirus vector of embodiment 16, wherein the genomic information encoding the infectious arenavirus viral vector is derived from the Junin virus Candid #1 strain.

Embodiment 16b is the arenavirus vector of any one of embodiments 1 to 16a, wherein the arenavirus is a lymphocytic choriomeningitis virus.

Embodiment 17 is a composition comprising the arenavirus vector of any one of embodiments 1-16b and a pharmaceutically acceptable carrier.

Embodiment 18 is a kit comprising the arenavirus vectors of any one of embodiments 1 to 16b or the composition of embodiments 17, and instructions for using the therapeutic combination in treating a hepatitis B virus (HBV) infection in a subject in need thereof.

Embodiment 19 is a method of treating a hepatitis B virus (HBV) infection in a subject in need thereof, comprising administering to the subject arenavirus vector of any one of embodiments 1 to 16b or the composition of any one of embodiments 18-19.

Embodiment 20 is the method of embodiment 19, wherein the treatment induces an immune response against a hepatitis B virus in a subject in need thereof, preferably the subject has chronic HBV infection.

Embodiment 21 is the method of embodiment 19 or 20, wherein the subject has chronic HBV infection.

Embodiment 21a is the method of any one of embodiments 19 to 21, wherein the subject is in need of a treatment of an HBV-induced disease selected from the group consisting of advanced fibrosis, cirrhosis and hepatocellular carcinoma (HCC).

Embodiment 21b is the method of any one of embodiments 19 to 21a, wherein the composition is administered by injection through the skin, e.g., intramuscular or intradermal injection, preferably intramuscular injection.

### EXAMPLES

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

### Example 1. HBV Core Plasmid & HBV Pol Plasmid

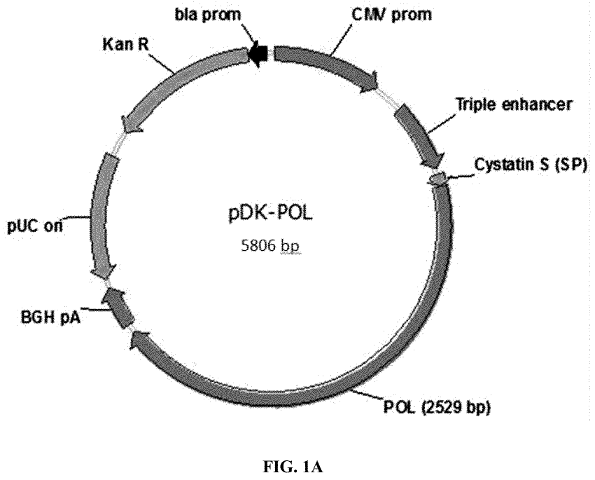

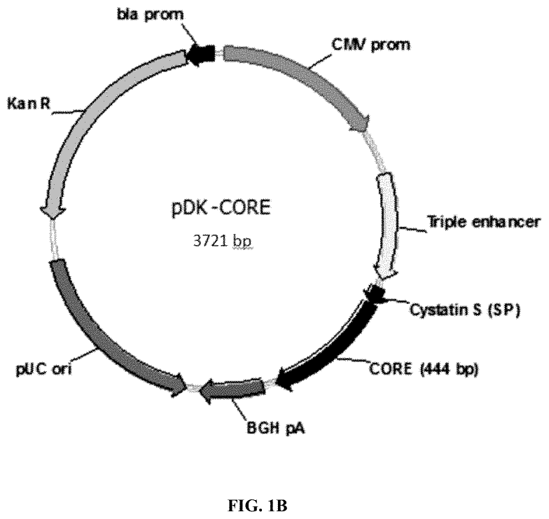

A schematic representation of the pDK-pol and pDK-core vectors is shown in FIG. 1A and 1B, respectively. An HBV core or pol antigen optimized expression cassette containing a CMV promoter (SEQ ID NO: 18), a splicing enhancer (triple composite sequence) (SEQ ID NO: 10), a coding sequence of Cystatin S precursor signal peptide SPCS (NP 0018901.1) (SEQ ID NO: 8), and pol (SEQ ID NO: 5) or core (SEQ ID NO: 1) gene was introduced into a pDK plasmid backbone, using standard molecular biology techniques.

The plasmids were tested in vitro for core and pol antigen expression by Western blot analysis using core and pol specific antibodies, and were shown to provide consistent expression profile for cellular and secreted core and pol antigens (data not shown).

### Example 2. Generation of Adenoviral Vectors Expressing a Fusion of Truncated HBV Core Antigen with HBV Pol Antigen

The creation of an adenovirus vector has been designed as a fusion protein expressed from a single open reading frame. Additional configurations for the expression of the two proteins, e.g. using two separate expression cassettes, or using a 2A-like sequence to separate the two sequences, can also be envisaged.

### Design of Expression Cassettes for Adenoviral Vectors

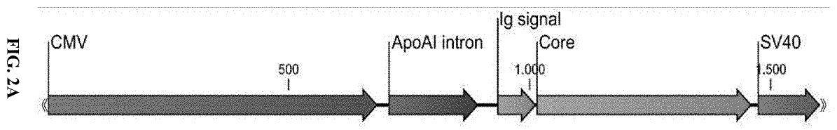

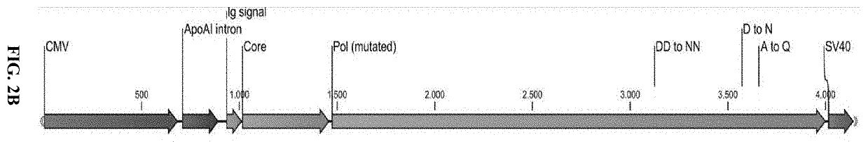

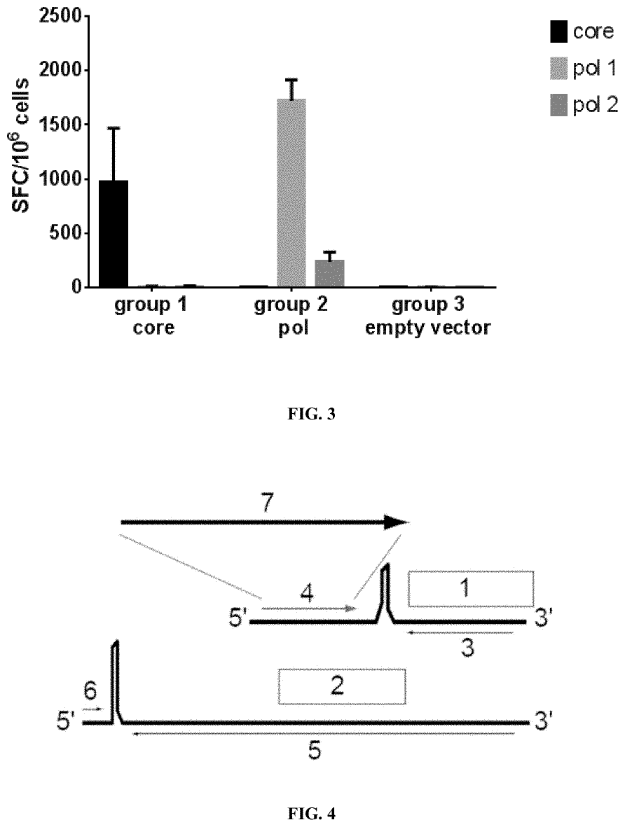

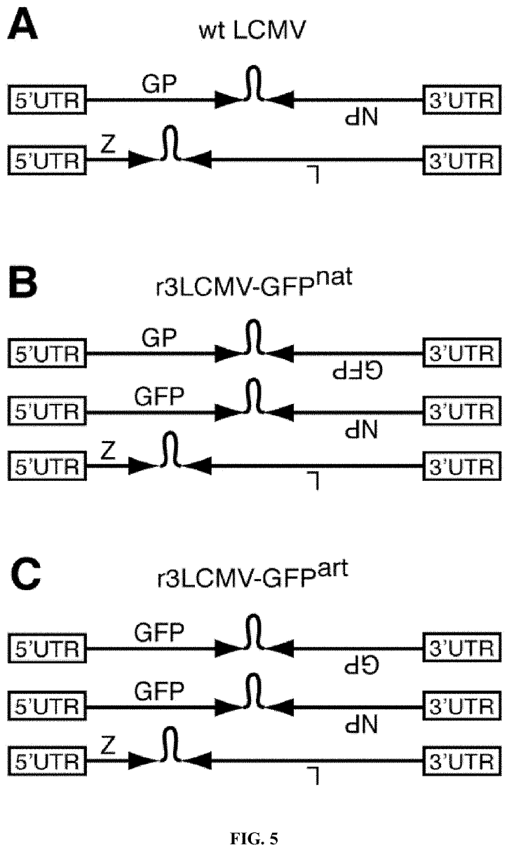

The expression cassettes (diagrammed in FIG. 2A and FIG. 2B) are comprised of the CMV promoter (SEQ ID NO: 19), an intron (SEQ ID NO:12) (a fragment derived from the human ApoAI gene—GenBank accession X01038 base pairs 295-523, harboring the ApoAI second intron), followed by the optimized coding sequence—either core alone or the core and polymerase fusion protein preceded by a human immunoglobulin secretion signal coding sequence (SEQ ID NO: 14), and followed by the SV40 polyadenylation signal (SEQ ID NO: 13).

A secretion signal was included because of past experience showing improvement in the manufacturability of some adenoviral vectors harboring secreted transgenes, without influencing the elicited T-cell response (mouse experiments).

The last two residues of the Core protein (VV) and the first two residues of the Polymerase protein (MP) if fused results in a junction sequence (VVMP) that is present on the human dopamine receptor protein (D3 isoform), along with flanking homologies.

The interjection of an AGAG linker between the core and the polymerase sequences eliminates this homology and returned no further hits in a Blast of the human proteome.

### Example 3. In Vivo Immunogenicity Study of DNA Vaccine in Mice

An immunotherapeutic DNA vaccine containing DNA plasmids encoding an HBV core antigen or HBV polymerase antigen was tested in mice. The purpose of the study was designed to detect T-cell responses induced by the vaccine after intramuscular delivery via electroporation into BALB/c mice. Initial immunogenicity studies focused on determining the cellular immune responses that would be elicited by the introduced HBV antigens.

In particular, the plasmids tested included a pDK-Pol plasmid and pDK-Core plasmid, as shown in FIGS. 1A and 1B, respectively, and as described above in Example 1. The pDK-Pol plasmid encoded a polymerase antigen having the amino acid sequence of SEQ ID NO: 7, and the pDK-Core plasmid encoding a Core antigen having the amino acid sequence of SEQ ID NO: 2. First, T-cell responses induced by each plasmid individually were tested. The DNA plasmid (pDNA) vaccine was intramuscularly delivered via electroporation to Balb/c mice using a commercially available TriGrid™ delivery system-intramuscular (TDS-IM) adapted for application in the mouse model in cranialis tibialis. See International Patent Application Publication WO2017172838, and U.S. patent application Ser. No. 62/607,430, entitled “Method and Apparatus for the Delivery of Hepatitis B Virus (HBV) Vaccines,” filed on Dec. 19, 2017 for additional description on methods and devices for intramuscular delivery of DNA to mice by electroporation, the disclosures of which are hereby incorporated by reference in their entireties. In particular, the TDS-IM array of a TDS-IM v1.0 device having an electrode array with a 2.5 mm spacing between the electrodes and an electrode diameter of 0.030 inch was inserted percutaneously into the selected muscle, with a conductive length of 3.2 mm and an effective penetration depth of 3.2 mm, and with the major axis of the diamond configuration of the electrodes oriented in parallel with the muscle fibers.

Following electrode insertion, the injection was initiated to distribute DNA (e.g., 0.020 ml) in the muscle. Following completion of the IM injection, a 250 V/cm electrical field (applied voltage of 59.4 -65.6 V, applied current limits of less than 4 A, 0.16 A/sec) was locally applied for a total duration of about 400 ms at a 10% duty cycle (i.e., voltage is actively applied for a total of about 40 ms of the about 400 ms duration) with 6 total pulses. Once the electroporation procedure was completed, the TriGri™ array was removed and the animals were recovered. High-dose (20 μg) administration to BALB/c mice was performed as summarized in Table 1. Six mice were administered plasmid DNA encoding the HBV core antigen (pDK-core; Group 1), six mice were administered plasmid DNA encoding the HBV pol antigen (pDK-pol; Group 2), and two mice received empty vector as the negative control. Animals received two DNA immunizations two weeks apart and splenocytes were collected one week after the last immunization.

TABLE 1
Mouse immunization experimental design of the pilot study.

| | | | Unilateral | | | | Endpoint |
|---|---|---|---|---|---|---|---|
| | | | Admin Site | | | | (spleen |
| | | | (alternate | | | Admin | harvest) |
| Group | N | pDNA | sides) | Dose | Vol | Days | Day |
| 1 | 6 | Core | CT + EP | 20 μg | 20 μL | 0, 14 | 21 |
| 2 | 6 | Pol | CT + EP | 20 μg | 20 μL | 0, 14 | 21 |
| 3 | 2 | Empty | CT + EP | 20 μg | 20 μL | 0, 14 | 21 |
| | | Vector | | | | | |
| | | (neg | | | | | |
| | | control) | | | | | |

CT, cranialis tibialis muscle; EP, electroporation.

Antigen-specific responses were analyzed and quantified by IFN-γ enzyme-linked immunospot (ELISPOT). In this assay, isolated splenocytes of immunized animals were incubated overnight with peptide pools covering the Core protein, the Pol protein, or the small peptide leader and junction sequence (2 μg/ml of each peptide). These pools consisted of 15 mer peptides that overlap by 11 residues matching the Genotypes BCD consensus sequence of the Core and Pol vaccine vectors. The large 94 kDan HBV Pol protein was split in the middle into two peptide pools. Antigen-specific T cells were stimulated with the homologous peptide pools and IFN-γ-positive T cells were assessed using the ELISPOT assay. IFN-γ release by a single antigen-specific T cell was visualized by appropriate antibodies and subsequent chromogenic detection as a colored spot on the microplate referred to as spot-forming cell (SFC).

Substantial T-cell responses against HBV Core were achieved in mice immunized with the DNA vaccine plasmid pDK-Core (Group 1) reaching 1,000 SFCs per 10⁶ cells (FIG. 8). Pol T-cell responses towards the Pol 1 peptide pool were strong (˜1,000 SFCs per 10⁶ cells). The weak Pol-2-directed anti-Pol cellular responses were likely due to the limited MHC diversity in mice, a phenomenon called T-cell immunodominance defined as unequal recognition of different epitopes from one antigen. A confirmatory study was performed confirming the results obtained in this study (data not shown).

The above results demonstrate that vaccination with a DNA plasmid vaccine encoding HBV antigens induces cellular immune responses against the administered HBV antigens in mice. Similar results were also obtained with non-human primates (data not shown).

It is understood that the examples and embodiments described herein are for illustrative purposes only, and that changes could be made to the embodiments described above without departing from the broad inventive concept thereof. It is understood, therefore, that this invention is not limited to the particular embodiments disclosed, but it is intended to cover modifications within the spirit and scope of the invention as defined by the appended claims.

1-19. (canceled)

20. An arenavirus vector comprising:
a non-naturally occurring polynucleotide sequence encoding a Hepatitis B virus (HBV) polymerase antigen consisting of an amino acid sequence that is at least 90% identical to SEQ ID NO: 7, wherein the HBV polymerase antigen does not have reverse transcriptase activity and RNase H activity and is capable of inducing a T cell response against at least HBV genotypes B, C, and D;
wherein the arenavirus vector is infectious, and wherein an open reading frame that encodes a glycoprotein of the arenavirus is deleted or functionally inactivated.

21. The arenavirus vector of claim 20, further comprising a non-naturally occurring polynucleotide sequence encoding a truncated HBV core antigen consisting of an amino acid sequence that is at least 95% identical to SEQ ID NO: 2 or SEQ ID NO: 4.

22. The arenavirus vector of claim 20, wherein the arenavirus vector is replication-deficient and has the ability to amplify and express its genetic information in infected cells but is unable to produce further infectious progeny particles in normal, not genetically engineered cells.

23. The arenavirus vector of claim 20, wherein the genomic information encoding the infectious arenavirus viral vector is derived from the lymphocytic choriomeningitis virus Clone 13 strain.

24. The arenavirus vector of claim 20, wherein the genomic information encoding the infectious arenavirus viral vector is derived from the lymphocytic choriomeningitis virus MP strain.

25. The arenavirus vector of claim 20, wherein the genomic information encoding the infectious arenavirus viral vector is derived from Junin virus.

26. The arenavirus vector of claim 20, wherein the viral vector comprises a genomic segment wherein the genomic segment comprises a nucleotide sequence that is at least 90% identical to the sequence of nucleotide 1639 to 3315 of SEQ ID NO: 29, or 1640 to 3316 of SEQ ID NO: 25.

27. The arenavirus vector of claim 20, wherein the viral vector comprises a genomic segment comprising a nucleotide sequence encoding an expression product whose amino acid sequence is at least 90% identical to the amino acid sequence encoded by 1639 to 3315 of SEQ ID NO: 29, or 1640 to 3316 of SEQ ID NO: 25.

28. The arenavirus vector of claim 20, wherein the HBV polymerase antigen consists of an amino acid sequence that is at least 98% identical to SEQ ID NO: 7.

29. The arenavirus vector of claim 20, further comprising a polynucleotide sequence encoding signal sequence operably linked to the N-terminus of the HBV polymerase antigen.

30. The arenavirus vector of claim 21, wherein:
a) the truncated HBV core antigen consists of the amino acid sequence of SEQ ID NO: 2 or SEQ ID NO: 4; and
b) the HBV polymerase antigen comprises the amino acid sequence of SEQ ID NO: 7.

31. The arenavirus vector of claim 30, wherein the non-naturally occurring polynucleotide sequence encoding the core antigen comprises the polynucleotide sequence of SEQ ID NO: 1 or SEQ ID NO: 3, and the non-naturally occurring polynucleotide sequence encoding the polymerase antigen comprises the polynucleotide sequence of SEQ ID NO: 5 or SEQ ID NO: 6.

32. The arenavirus vector of claim 21, encoding a fusion protein comprising the truncated HBV core antigen operably linked to the HBV polymerase antigen.

33. The arenavirus vector of claim 32, wherein the fusion protein comprises the truncated HBV core antigen operably linked to the HBV polymerase antigen via a linker.

34. The arenavirus vector of claim 33, wherein the linker comprises the amino acid sequence of (AlaGly)n, and n is an integer of 2 to 5.

35. The arenavirus vector of claim 34, wherein the fusion protein comprises the amino acid sequence of SEQ ID NO: 16.

36. A composition comprising the arenavirus vector of claim 20, and a pharmaceutically acceptable carrier.

37. An arenavirus vector comprising:
a non-naturally occurring polynucleotide sequence encoding a Hepatitis B virus (HBV) polymerase antigen consisting of an amino acid sequence that is at least 98% identical to SEQ ID NO: 7, wherein the HBV polymerase antigen does not have reverse transcriptase activity and RNase H activity and is capable of inducing a T cell response against at least HBV genotypes B, C, and D;
wherein the arenavirus vector is infectious, and wherein an open reading frame that encodes a glycoprotein of the arenavirus is deleted or functionally inactivated.

38. The arenavirus vector of claim 37, further comprising a non-naturally occurring polynucleotide sequence encoding a truncated HBV core antigen consisting of an amino acid sequence that is at least 95% identical to SEQ ID NO: 2 or SEQ ID NO: 4

39. A method of treating or preventing a hepatitis B virus (HBV) infection in a subject in need thereof, comprising administering to the subject the arenavirus vector of claim 20.
